(19) United States
(12) Patent Application Publication
(10) Pub. No.: US 2020/0297434 A1
(43) Pub. Date: Sep. 24, 2020

(54) ROBOT FOR INSERTION OF AN ELONGATE FLEXIBLE MEDICAL INSTRUMENT AND ASSOCIATED ACCESSORIES

(71) Applicant: ROBOCATH, Rouen (FR)

(72) Inventors: Philippe BENCTEUX, Bois-Guillaume (FR); Sébastien DEBOEUF, Herblay (FR); Fabien DESTREBECQ, Bourgtheroulde (FR); Julien MAUREL, Montjavoult (FR); Marc BOULANGE, Gruchet-Saint-Simeon (FR); Bruno FOURNIER, Saint Ouen (FR)

(21) Appl. No.: 16/086,171

(22) PCT Filed: Mar. 10, 2017
(86) PCT No.: PCT/FR2017/050545

§ 371 Date: Sep. 18, 2018

(30) Foreign Application Priority Data
Mar. 18, 2016 (FR) 16 52304

(51) Int. Cl. A61B 34/30

(52) U.S. Cl. CPC A61B 34/30; A61B 2017/00477

(57) ABSTRACT

A robot for insertion of an elongate flexible medical instrument into a patient includes drive modules for driving the elongate flexible medical instrument in this patient. A drive module for an elongate flexible medical instrument transmits to the instrument a movement of translation and/or a movement of rotation which may be subject to a slack effect. The main aim of the invention is to reduce this slack effect by acting at several locations along the transmission chain between the distal end of the elongate flexible medical instrument, the last section to undergo the rotational movement originally imparted by the user at a human-machine control interface of the drive module. The advancement of the elongate flexible medical instrument is thus rendered more effective for the process of this advancement, safer for the patient, and more ergonomic for the practitioner who is the user of the elongate flexible medical instrument.

### FIELD OF INVENTION

The invention relates to the field of robots for insertion of an elongate flexible medical instrument, guide, or other elongate flexible medical instrument of elongate flexible medical instrument drive modules included in these robots for insertion of an elongate flexible medical instrument, and accessories associated with these robots for insertion of an elongate flexible medical instrument. These accessories include, in particular, elongate flexible medical instrument drive systems with a non-motorized linear rail and with an elongate flexible medical instrument motorized drive module, elongate flexible medical instrument drive systems with a motorized linear rail and with an elongate flexible medical instrument drive module, the sterile barriers between consumable and non-consumable parts in robots for insertion of an elongate flexible medical instrument, the elongate flexible medical instrument guide tracks in robots for insertion of an elongate flexible medical instrument, the articulated arms carrying the drive module in robots for insertion of an elongate flexible medical instrument, the arterial introducer connectors in robots for insertion of an elongate flexible medical instrument, the guide rollers in robots for insertion of an elongate flexible medical instrument, and the remote control stations, for robots for insertion of an elongate flexible medical instrument, which incorporate protective shields.

### BACKGROUND OF INVENTION

A robot for insertion of an elongate flexible medical instrument into a patient includes a module to drive the insertion of the elongate flexible medical instrument into the patient. The module to drive the insertion of the elongate flexible medical instrument transmits, to the elongate flexible medical instrument, a translational movement and/or rotational movement which may possibly be combined.

The transmission of this translational movement and/or rotational movement should be controlled to obtain regular and efficient advancement of the elongate flexible medical instrument in the patient. As this transmission of movement occurs over a certain distance, control over this transmission is neither immediate nor automatic.

In particular, when a rotational movement about its axis is imparted to an elongate flexible medical instrument at an intermediate portion thereof, this rotational movement is not immediately and regularly transmitted to its distal end (patient side).

On the contrary, due to the friction, difficult passages, and curves experienced by this elongate flexible medical instrument as it advances, stored energy is released abruptly and in spurts after a delay, resulting in a jerky rotational movement at its distal end. In response to a rotation at constant speed that is imparted by the robot at the proximal end of the elongate flexible medical instrument (insertion robot side), an irregular rotational speed at the distal end of the elongate flexible medical instrument is found.

This slack effect is both annoying and disruptive for the user of the elongate flexible medical instrument drive module. This slack effect may even be amplified by using a winder for the elongate flexible medical instrument.

This slack effect will be exacerbated by any eccentricities of the elongate flexible medical instrument, whether geometric or concerning density variations in the material. Such eccentricities of the elongate flexible medical instrument will increase the contrast between the areas of easier rotation and areas of the more difficult rotation, accentuating the jerkiness in the transmission of rotational movement between the human-machine control interface and the distal end of the elongate flexible medical instrument. In practice, this causes “jumps” in the rotation which increases the difficulty in controlling the angle of rotation.

This slack effect is also aggravated by twists and curves in the path followed by the elongate flexible medical instrument as it advances.

The main aim of the invention is to reduce this slack effect by acting at different locations along the transmission chain between the distal end of the elongate flexible medical instrument, which is the last portion of the elongate flexible medical instrument to which is imparted the rotational movement originally imparted by the user at a human-machine control interface of the elongate flexible medical instrument drive module.

This advantageously avoids exacerbating the slack effect or at least exacerbating it very little, even in comparison to manual manipulation which benefits directly from all the practitioner's dexterity.

The advancement of the elongate flexible medical instrument is thus rendered more effective for the process of such advancement of the elongate flexible medical instrument, safer for the patient, and more ergonomic for the practitioner who is the user of the elongate flexible medical instrument.

To achieve this, various tools and accessories included in the robot for insertion of an elongate flexible medical instrument are available to the practitioner, to help with controlling this advancement of the elongate flexible medical instrument in the patient.

### SUMMARY OF THE INVENTION

The object of the present invention is to provide tools and accessories included in the robot for insertion of an elongate flexible medical instrument, which at least partially overcome the above disadvantages.

More particularly, the invention aims to supply tools and accessories included in the robot for insertion of an elongate flexible medical instrument, provided to the practitioner to help with controlling this advancement of the elongate flexible medical instrument in the patient.

A first object of the invention concerns an interaction, in the robot for insertion of an elongate flexible medical instrument, between the drive module and the rail on which it slides, based on a non-motorized linear rail and a motorized drive module. To improve management of the slack effect, this interaction eliminates the winder conventionally used for winding the elongate flexible medical instrument and which amplifies the slack effect. However, the absence of a winder results in a significant path length for the drive module on the linear rail, with the issue of managing sterility along the entire path. For the skilled person, this sterility management along the entire path is a deterrent, dissuading him or her from eliminating the winder.

To this end, this first object of the invention provides an elongate flexible medical instrument drive system of a robot for insertion of an elongate flexible medical instrument, comprising: an arm, a non-motorized linear rail carried by the arm, and an elongate flexible medical instrument motorized drive module which slides along the linear rail.

According to preferred embodiments, the invention comprises one or more of the following features which may be used separately or in partial combination or in full combination, not only with the object of the invention mentioned above, but with all objects of the invention mentioned in the rest of this application.

Preferably, the elongate flexible medical instrument motorized drive module comprises two parts which can be detached from one another: a reusable motor without contact with the linear rail, and a disposable carriage sliding on the linear rail, this carriage preferably being for one-time use.

Thus, only the simplest and least expensive part of the drive module in contact with the elongate flexible medical instrument which in turn is in contact with the patient, will have to be discarded. In contrast, the most complex and expensive part of the drive module, which is not in direct contact with the elongate flexible medical instrument or in indirect contact with the patient, will be retained.

Preferably, the sliding of the disposable carriage on the linear rail provides the translational movement of the elongate flexible medical instrument.

Thus, the simple movement of the drive module on the linear rail automatically achieves, with no additional components, one of the four desired movements which are the translation and/or rotation of the elongate flexible medical instrument.

Preferably, the disposable carriage comprises a contact surface with the linear rail, this contact surface being E-shaped so that the disposable carriage rests on three of the four sides of the linear rail.

The drive module is thus well supported, guided, and held by the linear rail if it moves along the top of the linear rail, and is well supported, guided, and held by the linear rail even if it is moving along the side or below the linear rail.

Preferably, the linear rail is disposable, and preferably the linear rail is for one-time use.

The sterility issue is thus handled with maximum efficiency and for a reasonable cost, because the disposable rail here is a fairly simple and inexpensive element since the motorization has been completely removed from it.

Preferably, the drive system also comprises a consumable sterile barrier between the reusable motor and the disposable carriage which are integrally secured together.

Sterility is thus more effectively ensured, even in the sensitive area that forms the boundary between the consumable and non-consumable parts of the drive module.

Preferably, the sterile barrier comprises a plate which is perforated to allow the passage of couplings between the disposable carriage and reusable motor and which is surrounded by a film attached to the edges of the plate.

The risk of damaging the sterile barrier in the region of high mechanical stress that forms the boundary between the consumable and non-consumable parts of the drive module is thus reduced.

Preferably, this sterile barrier includes the disposable carriage which is surrounded by a film attached to the edges of the disposable carriage.

The assembly formed by the consumable part of the drive module and by the sterile barrier is thus a more compact entity of smaller footprint, which can be more easily removed after patient diagnosis and discarded as one unit with a reduced risk of contaminating other elements which may be either the non-consumable part of the drive module or the non-consumable part of the drive module and the sterility barrier to be put in place for diagnosis of the next patient.

Preferably, the drive system also comprises another consumable sterile barrier encompassing the entire arm, but neither the linear rail nor the elongate flexible medical instrument drive module.

Thus, the consumable part of the drive module is completely isolated, in a sterile manner, not only from the non-consumable part of the drive module but also from the rest of the drive system which is also non-consumable.

Preferably, the path length of the motorized module along the linear rail is between 60 cm and 120 cm.

A significant path length for the drive module is thus maintained despite the absence of the winder.

Preferably, the linear rail comprises at least one groove guiding the elongate flexible medical instrument.

The elongate flexible medical instrument is thus carried and driven by the drive module while being guided along its largest portion by the linear rail. The drive module and the linear rail work together to guide the elongate flexible medical instrument along its entire length.

Preferably, the groove is closed by a cover which opens when the motorized module passes by and which closes after the passage of the motorized module.

The elongate flexible medical instrument thus remains better protected, even outside the drive module driving area.

Preferably, the arm comprises: a post that is movable in vertical translation and in horizontal translation, and two bars forming a V, the apex of the V preferably comprising a ball joint coupling it to the top of the movable post, the free ends of the V preferably being fixedly connected to the linear rail.

The arm structure is thus able to bring the drive module closer to the arterial introducer placed in the patient, then enabling the entire travel of the elongate flexible medical instrument in the patient solely with the linear path of the linear rail.

Preferably, the drive system also comprises locking members for locking the assembly formed by the arm, linear rail, and motorized module, such that this assembly can be moved as a unit relative to the operating table.

This can save time in adjusting the positions of the various elements of the arm during an intervention.

Preferably, the motorized drive module contains a catheter drive module and a guide drive module for driving in translation and in rotation.

The movement of the guide can thus be decoupled from that of the catheter, allowing the guide to fully fulfill its role by sending it in front of the catheter, passing through and traversing all the difficult areas, then allowing the catheter to advance smoothly in a regular and effective manner. Preferably, the motorized drive module for an elongate flexible medical instrument is controlled by a wireless link and/or has one or more electric batteries as the primary and preferably exclusive source of energy.

Its maneuverability and autonomy are thus enhanced.

Preferably, the elongate flexible medical instrument is a catheter and/or a guide.

A second object of the invention concerns, in the robot for insertion of an elongate flexible medical instrument, an interaction between the drive module and the rail on which it slides, based on a motorized linear rail and a non-motorized drive module. To improve management of the slack effect, this interaction eliminates the winder conventionally used for winding the elongate flexible medical instrument and which amplifies the slack effect. However, the absence of a winder results in a significant path length for the drive module on the linear rail, with the issue of managing sterility along the entire path. For the skilled person, this sterility management along the entire path is a deterrent, dissuading him or her from eliminating the winder. The consumable part of the drive system assembly is less important than in the case of the first object of the invention, because there is now only a portion of the drive module and the sterility barrier, with exclusion of the linear rail.

However, this motorization of the linear rail is somewhat more complex than motorization of the drive module alone as was the case in the first object of the invention.

To this end, this second object of the invention provides an elongate flexible medical instrument drive system of a robot for insertion of an elongate flexible medical instrument, comprising: an arm, a motorized linear rail carried by the arm, an elongate flexible medical instrument drive module which slides along the linear rail under the effect of the motorization of only the linear rail.

According to preferred embodiments, the invention comprises one or more of the following features which may be used separately or in partial combination or in full combination, not only with the object of the invention mentioned above, but with all objects of the invention mentioned in the rest of this application.

Preferably, the elongate flexible medical instrument drive module comprises two parts which can be detached from one another: a reusable carriage sliding on the linear rail, and a disposable support without contact with the linear rail, said support preferably being for one-time use, driving the elongate flexible medical instrument.

Thus, only the simplest and least expensive part of the drive module in contact with the elongate flexible medical instrument which in turn is in contact with the patient, will have to be discarded. In contrast, the most complex and expensive part of the drive module, which is not in direct contact with the elongate flexible medical instrument or in indirect contact with the patient, will be retained.

Preferably, the drive system also comprises a consumable sterile barrier passing between the reusable carriage and the disposable support which are integrally secured together.

Sterility is thus more effectively ensured, even in the sensitive area that forms the boundary between the consumable and non-consumable parts of the drive module.

Preferably, the consumable sterile barrier between the reusable carriage and the disposable support which are integrally secured together, also encompasses the entire arm.

Thus, the consumable part of the drive module is completely isolated, in a sterile manner, not only from the non-consumable part of the drive module but also from the rest of the drive system which is also non-consumable, and this is achieved using a single sterile barrier and not two sterile barriers as in the first object of the invention.

Preferably, the motorized drive module comprises a catheter drive module and a guide drive module for driving in translation and in rotation. The movement of the guide can thus be decoupled from that of the catheter, allowing the guide to fully fulfill its role by sending it in front of the catheter, passing through and traversing all the difficult areas, then allowing the catheter to advance smoothly in a regular and effective manner.

Preferably, the elongate flexible medical instrument is a catheter and/or a guide.

A third object of the invention concerns preserving the sterility of an interaction between the drive module and the rail on which it slides, or an interaction between the drive module and the robotic arm at the end of which it advances, in the robot for insertion of an elongate flexible medical instrument. To improve management of the slack effect, this interaction eliminates the winder conventionally used for winding the elongate flexible medical instrument and which amplifies the slack effect. However, the absence of a winder results in a significant path length for the drive module on the linear rail or at the end of the robotic arm, with the issue of managing sterility along the entire path. For the skilled person, this sterility management along the entire path is a deterrent, dissuading him or her from eliminating the winder. This third object of the invention facilitates and improves the management of this sterility.

To this end, this third object of the invention provides a method for creating a sterile barrier between the consumable and non-consumable parts of an elongate flexible medical instrument drive system of a robot for insertion of an elongate flexible medical instrument, comprising a step of installing a consumable sterile skirt separating a linear rail from at least a portion of an elongate flexible medical instrument drive module in this elongate flexible medical instrument drive system.

To this end, this third object of the invention also provides a consumable sterile skirt adapted to separate a linear rail from at least a portion of an elongate flexible medical instrument drive module in an elongate flexible medical instrument drive system of a robot for insertion of an elongate flexible medical instrument, thereby providing a sterile barrier between the consumable and non-consumable parts of this elongate flexible medical instrument drive system.

To this end, this third object of the invention also provides an elongate flexible medical instrument drive system of a robot for insertion of an elongate flexible medical instrument, comprising a sterile barrier between its consumable and non-consumable parts, comprising: a linear rail, an elongate flexible medical instrument drive module, and a consumable sterile skirt separating the linear rail from at least a portion of the elongate flexible medical instrument drive module.

According to preferred embodiments, the invention comprises one or more of the following features which may be used separately or in partial combination or in full combination, not only with the object of the invention mentioned above, but with all objects of the invention mentioned in the rest of this application.

Preferably, the skirt is longitudinally wrinkled, on each side of the elongate flexible medical instrument drive module, so as to maintain the sterility barrier for the entire translational path of the elongate flexible medical instrument drive module along the linear rail.

The wrinkled portions, by elongating then retracting in phase opposition relative to one another, allow maintaining sufficient sterile coverage on each side of the drive module for all positions of the drive module, along the entire translational path. This manner of ensuring sterile protection by allowing the skirt to absorb and compensate for movements of the drive module is particularly simple and effective.

Preferably, the wrinkled skirt comprises lateral elastic members to hold this wrinkled skirt around the linear rail.

Preferably, the skirt is longitudinally wrinkled, on each side of a central portion corresponding to an attachment at the elongate flexible medical instrument drive module, so as to maintain the sterility barrier for the entire translational path of the elongate flexible medical instrument drive module along the linear rail, the wrinkled skirt advantageously comprising lateral elastic members to hold this wrinkled skirt around the linear rail.

The skirt is thus better retained around the rail, reducing or eliminating the chance of the skirt falling to one side or the other of the rail.

Preferably, the skirt is longitudinally slit while having one side of the slit overlap the other side of the slit so as to maintain a sterility channel around the linear rail.

Sterility is thus guaranteed with no significant risk of dust entering between the two overlapping portions of the skirt. However, this covering of one portion of the skirt by the other is somewhat complex to manipulate and requires a certain rigidity of the skirt material so that it is not spontaneously opened by its own weight.

Preferably, the slit is opened in response to a spreading front shape of the elongate flexible medical instrument drive module and is closed in response to a closing rear shape of the elongate flexible medical instrument drive module.

This allows the passage of the drive module while sterility is continuously maintained for the major portion of the path, only the portion of the path corresponding to the passage of the drive module being temporarily or even briefly open during the passage of the module.

Preferably, the skirt is longitudinally slit with the sides of the slit lying edge-to-edge, to maintain a sterility channel around the linear rail.

The skirt thus closes in a simple manner while ensuring adequate sterility. However, the connection with the drive module occurs beneath the linear rail, which is a little trickier to manage in order to avoid the entry of dust into the wrong areas.

Preferably, the slit is opened in response to a spreading front shape of the elongate flexible medical instrument drive module and is closed in response to a closing rear shape of the elongate flexible medical instrument drive module.

Thus, it is the actual movement of the drive module which ensures that the skirt opens in front of it and then closes behind it, without requiring the intervention or use of some other additional opening and/or closing element.

Preferably, the elongate flexible medical instrument drive system of a robot for insertion of an elongate flexible medical instrument also comprises a pouch surrounding the elongate flexible medical instrument drive module.

Sterility is thus ensured by two separate elements, one ensuring the sterility of the rail only, and the other ensuring the sterility of the drive module only.

Preferably, the elongate flexible medical instrument drive system of a robot for insertion of an elongate flexible medical instrument also comprises a first winder/unwinder integrally secured to a first end of the elongate flexible medical instrument drive module, a first consumable sterile skirt immobilized on one side relative to a first end of the linear rail and located on another side inside the first winder/unwinder so as to be able to respectively wind or unwind depending on the direction of movement of the elongate flexible medical instrument drive module along the linear rail, the first consumable sterile skirt being integrally secured on one side to the first end of the linear rail, a second winder/unwinder integrally secured to a second end of the elongate flexible medical instrument drive module, a second consumable sterile skirt immobilized on one side relative to a second end of the linear rail and located on another side in the second winder/unwinder so as to be able to respectively wind or unwind while the first consumable sterile skirt unwinds or winds, the second consumable sterile skirt being integrally secured on one side to the second end of the linear rail.

Sterility is thus ensured, because there is no hole in the sterility barrier, even a small one, even temporary or brief. However, the use of two winders makes this sterility-ensuring device relatively complex and expensive.

Preferably, the consumable sterile skirt has a length which is at least twice the path length of the elongate flexible medical instrument drive module along the linear rail.

Preferably, the consumable sterile skirt is smooth over its entire surface.

This type of skirt thus has a very simple structure. The required flexibility is provided by an excess of material, although this can become significant.

Preferably, the elongate flexible medical instrument drive system for a robot for insertion of an elongate flexible medical instrument also comprises an arterial introducer, a cowl located on the side closest to the arterial introducer, arranged so as to bring the consumable sterile skirt from the opposite side relative to an elongate flexible medical instrument drive module with respect to the sliding plane of the linear rail.

The presence of the cowl prevents interference from the excess skirt material at the arterial introducer when it is no longer used to cover the path of the drive module, in other words when the drive module arrives at the arterial introducer.

Preferably, the consumable sterile skirt is fixed to the linear rail, and the elongate flexible medical instrument drive system comprises another consumable sterile skirt that covers both the linear rail and the elongate flexible medical instrument drive module.

Thus, a good degree of sterility can be ensured due to this double level of sterile protection. However, this sterility-ensuring system is relatively bulky and expensive, as each skirt takes up space and also must be removed and replaced after each patient examination.

Preferably, the consumable sterile skirt is arranged around the linear rail so as to be rotated about the linear rail, about an axis perpendicular to the longitudinal axis of the linear rail, when the elongate flexible medical instrument drive module moves along the linear rail.

The skirt is then relatively simple in structure. However, precise dimensions are required to allow the skirt to slide smoothly when accompanying the movement of the drive module, otherwise there is a slight risk of it catching which is to be avoided.

Preferably, the consumable sterile skirt is weighted so as to remain around the linear rail.

The skirt opening is thus kept at the bottom by gravity, improving the preservation of sterility.

Preferably, the elongate flexible medical instrument drive system of a robot for insertion of an elongate flexible medical instrument also comprises flanges to guide the rotation of the consumable sterile skirt around the linear rail.

The skirt opening is thus kept at the bottom by the guidance of the guide flanges, thereby improving the preservation of sterility.

Preferably, the elongate flexible medical instrument is a catheter and/or a guide.

A fourth object of the invention concerns, in the robot for insertion of an elongate flexible medical instrument, the protection of an interaction between the drive module and the rail on which it slides, or an interaction between the drive module and the robotic arm at the end of which it operates, in particular the protection of the elongate flexible medical instrument in the area between the drive module and the arterial introducer. To improve management of the slack effect, this interaction eliminates the winder conventionally used for winding the elongate flexible medical instrument and which amplifies the slack effect. However, the absence of a winder results in a significant path length for the drive module on the linear rail or at the end of the robotic arm, with the issue of protecting the elongate flexible medical instrument over the entire area located between the drive module and the arterial introducer. For the skilled person, protecting this area for this entire path length is a deterrent, dissuading him or her from eliminating the winder. This fourth object of the invention facilitates and improves the protection of this area located between the drive module and the arterial introducer.

To this end, the fourth object of the invention provides an elongate flexible medical instrument drive system, comprising: an elongate flexible medical instrument, an elongate flexible medical instrument drive module whose movement causes movement of the elongate flexible medical instrument, preferably by pushing the elongate flexible medical instrument, an arterial introducer, and a track to guide the elongate flexible medical instrument between the elongate flexible medical instrument drive module and the arterial introducer.

According to some preferred embodiments, the invention comprises one or more of the following features which can be used separately or in partial combination or in full combination, not only with the object of the invention mentioned above, but with all objects of the invention mentioned in the rest of this application.

Preferably, the guide track is structured to open and then preferably to close when the elongate flexible medical instrument drive module passes by.

Thus, the elongate flexible medical instrument is better retained, because it is only during the passage of the drive module and at this drive module that there is an opening in the guide track for the elongate flexible medical instrument; indeed, elsewhere and the rest of the time, the guide track remains closed, and the elongate flexible medical instrument is held in place with no risk of exiting the guide track.

Preferably, the guide track is a slit tube.

The elongate flexible medical instrument is thus held in place by means of a simple structure.

Preferably, the guide track is closed by a slide fastener or zipper.

The elongate flexible medical instrument is thus held in place by a slightly more efficient structure.

Preferably, the guide track is a section which is flexible when open and rigid when refolded and closed.

Thus the elongate flexible medical instrument is held in place by a robust and efficient structure. This embodiment is a particularly good compromise between relative simplicity of the structure and high efficiency combined with high reliability.

Preferably, the section comprises: a bottom, two longitudinal side members respectively connected to the bottom and pivotally connected to this bottom, and two closure members respectively located on the two longitudinal side members, able to engage each other to close the section, the closed cavity of the section then being bounded by the bottom, the two longitudinal side members, and the two closure members.

Thus the elongate flexible medical instrument is held in place by an actual channel with rigid walls protecting the elongate flexible medical instrument.

Preferably, the cross-sectional dimensions of the side members and bottom of the section are determined so that the section is self-supporting when the closure members are closed, and the section is not self-supporting when the closure members are open. The self-supporting character of the section means that this section retains a linear shape or at least only bends to a limited extent when supported by its two ends.

Thus the elongate flexible medical instrument is held in place by a structure which does not sag excessively downwards when it is open and the elongate flexible medical instrument is no longer inside. The footprint of this structure for retaining the elongate flexible medical instrument is therefore reduced.

Preferably, the closure members are coupled by clipping one to the other.

The elongate flexible medical instrument is thus held in place by a simple and effective structure, enabling rapid opening and closing which can occur many times without damaging the structure.

Preferably, the pivoting connections are thinned areas in the material.

Thus, the elongate flexible medical instrument is held in place by a structure that, in addition to being robust, is relatively flexible to allow an opening and a closing which are both fast and wide, while still holding the elongate flexible medical instrument in place. In addition, this flexibility allows folding it into segments, facilitating disposal.

Preferably, the thinned areas in the material are notches which each have parallel beveled edges.

Thus, once the section is returned to the closed position, the walls of the section are rigid, almost as if there were no such thinned areas.

Preferably, each notch has a wide bottom relative to the width between the beveled edges.

Thus, despite numerous openings and closings at these thinned areas, there will be no premature wear which could eventually result in premature failure of the walls of this section at the bottom of these thinned areas.

Preferably, the guide track is in the form of a drag chain having a longitudinal opening which has a smaller width than the diameter of the elongate flexible medical instrument and which is flexible but asymmetrical to allow the elongate flexible medical instrument to more easily enter than exit.

Thus, the guide track is particularly strong and protective of the elongate flexible medical instrument placed inside, but the structure of the guide track is relatively complex.

Preferably, the guide track has the shape of a spiral winding around the elongate flexible medical instrument, this spiral being rotatable about the elongate flexible medical instrument.

The guide track thus has a relatively simple structure, but guiding the elongate flexible medical instrument by rotation of the spiral may be a little more difficult.

Preferably, the guide track comprises two parts which are fixed at one end to the elongate flexible medical instrument drive module, are respectively fixed at the other end to the interior of two winders, and form a single band outside these two winders within which they respectively wind as the elongate flexible medical instrument drive module slides towards the arterial introducer.

The guide track thus has the advantage of shrinking as the drive module advances, and therefore to be temporarily less bulky. However, this is achieved at the cost of a relatively high complexity of the guide track structure, due to the presence of two winders.

Preferably, the guide track comprises two rectangular crenellated parts which are flexible when separated from one another and form a rigid channel of rectangular cross-section when fitted one into the other.

The guide track is thus relatively robust in the closed position and relatively flexible in the open position, which is of interest. However, here again, this guide track has a certain complexity and requires lower manufacturing tolerances to ensure proper nesting of the crenellations.

Preferably, the guide track is in the form of a bellows.

The guide track is thus robust and of a length which changes with the movement of the drive module. However, this guide track is relatively cumbersome and only ensures a limited range for the path of travel of the drive module.

Preferably, the guide track comprises: an open and rigid guide channel in the recess of which the elongate flexible medical instrument is to be placed, a flexible cover which is fixed at one end to the elongate flexible medical instrument drive module and which is fixed at the other end to the interior of a winder into which it is wound as the elongate flexible medical instrument drive module slides towards the arterial introducer.

Thus, the cover of the guide track has the advantage of shrinking as the drive module advances, and therefore to be temporarily less bulky. The guide channel meanwhile remains of fixed length. However, this is achieved at the cost of relative complexity in the guide track structure, due to the presence of a winder.

Preferably, the elongate flexible medical instrument is a catheter and/or a guide.

A fifth object of the invention concerns, in the robot for insertion of an elongate flexible medical instrument, an interaction between the drive module and the robotic arm at the end of which the drive module operates. To improve management of the slack effect, this interaction eliminates the winder conventionally used for winding the elongate flexible medical instrument and which amplifies the slack effect. The absence of a winder here results in a smaller path of travel than when using a linear rail on which the drive module slides. However, although the problem of sterility management along the entire path of travel of the drive module is facilitated, it is at the cost of increasing the mechanical complexity of the support for the drive module; Indeed, the robotic arm has a number of joints, which could deter the skilled person from eliminating the winder for the elongate flexible medical instrument.

To this end, the fifth object of the invention provides an elongate flexible medical instrument drive system of a robot for insertion of an elongate flexible medical instrument, comprising: an articulated arm comprising at least three segments pivotally connected together and robotized, which is able to trace a linear path through space at its distal end, and an elongate flexible medical instrument drive module integrally secured to this distal end.

According to some preferred embodiments, the invention comprises one or more of the following features which can be used separately or in partial combination or in full combination, not only with the object of the invention mentioned above, but with all objects of the invention mentioned in the rest of this application.

Preferably, the spatial orientation of the elongate flexible medical instrument drive module is kept constant during its movement along said linear path.

Thus, controlling the movement of the elongate flexible medical instrument drive module is easier and the movement of the elongate flexible medical instrument drive module is smoother.

Preferably, this linear path through space remains within a horizontal plane, in other words in a plane parallel to the plane of the examination table. The linear path can then either be perfectly horizontal or have a slight angle to the examination table.

Thus, movement of the drive module is more easily controllable and manageable.

Preferably, the arm comprises at least four segments pivotally connected together, preferably only four segments pivotally connected together.

Thus, more degrees of freedom are available to enable the drive module to be moved as desired.

Preferably, the elongate flexible medical instrument drive system of a robot for insertion of an elongate flexible medical instrument further comprises: an adjustment rail carrying the proximal end of this arm, and a device for locking this proximal end of the arm on the adjustment rail during linear movement of this distal end.

Thus, the overall movement of the drive module is split into two movements. To begin with, there is a first positioning movement of the drive module in order to place the drive module in the proper position for starting its travel, this proper starting position varying according to the patient's morphology and the chosen point of entry into the patient for the elongate flexible medical instrument. Next, there is a second movement to advance the drive module along its path of travel, corresponding to the advancement of the elongate flexible medical instrument in the patient. The adjustment rail is responsible for the first movement to position the drive module, while the robotic arm is responsible for the second movement to advance the drive module. This division into two movements, first the movement to position the drive module and then the movement to advance the drive module, allows better management and optimization of each of these two distinct movements. In addition, a reasonable amount of motor power is required.

Preferably, the adjustment rail rests on an examination table, preferably being fixed to said examination table.

Thus, any relative movements between the examination table and the floor do not need to be compensated for, because they have no impact on the advancement of the elongate flexible medical instrument within the patient.

Preferably, the proximal end of the arm rests on an examination table, pivotally connected by a rotary connection to said examination table, advantageously pivotally connected only by this rotary connection to the examination table.

The overall structure of the elongate flexible medical instrument drive system of a robot for insertion of an elongate flexible medical instrument is thus simplified. However, the magnitude of the degrees of freedom of movement is relatively small unless the robotic arm has long and robust segments.

Preferably, the elongate flexible medical instrument drive system of a robot for insertion of an elongate flexible medical instrument further comprises: an adjustment rail carrying a non-articulated support post to which the proximal end of said arm is integrally secured, and a device for locking this support post on the adjustment rail during linear movement of this distal end.

The required motor power is thus quite low, particularly due to the raised elevation obtained by the support post. However, the number of components and subassemblies is relatively high, significantly increasing the overall complexity of the elongate flexible medical instrument drive system of a robot for insertion of an elongate flexible medical instrument.

Preferably, the adjustment rail rests on an examination table, preferably being fixed to said examination table.

Thus, any relative movements between the examination table and the floor do not need to be compensated for because they have no effect on the advancement of the elongate flexible medical instrument within the patient.

Preferably, the elongate flexible medical instrument drive system of a robot for insertion of an elongate flexible medical instrument further comprises: a non-articulated support post resting on the floor, to which the proximal end of said arm is integrally secured, and a device slaving it to the movement of an operating table during linear movement of this distal end.

Preferably, the support post rests on the floor.

Thus, the elongate flexible medical instrument drive system of a robot for insertion of an elongate flexible medical instrument is rendered completely independent of the examination table. However, relative movements between the examination table and the elongate flexible medical instrument drive system of a robot for insertion of an elongate flexible medical instrument must be compensated for.

Preferably, the support post is higher than the examination table associated with the elongate flexible medical instrument drive system.

The motor power required thus becomes lower, because the force required to counteract gravity is lower.

Preferably, the elongate flexible medical instrument drive system of a robot for insertion of an elongate flexible medical instrument further comprises: an articulated adjustment arm carrying the proximal end of said robotic arm, and a device for locking this articulated adjustment arm during linear movement of this distal end.

Less motor power is then required to move the drive module along its path, due to the high number of degrees of freedom in the device for positioning the drive module before starting its path of movement.

Preferably, the articulated adjustment arm rests on an examination table, preferably being fixed to the examination table.

Thus, any relative movements between the examination table and floor no longer need to be compensated for, because they no longer impact the advancement of the elongate flexible medical instrument within the patient.

Preferably, the articulated adjustment arm comprises at least three segments pivotally connected together.

Great freedom of movement is thus given to the positioning device, which can therefore more easily bring the drive module to the desired position, right next to the arterial introducer.

Preferably, the elongate flexible medical instrument drive system of a robot for inserting an elongate flexible medical instrument further comprises a post to which is integrally secured the proximal end of said articulated robotic arm, and all segments of the articulated robotic arm only deploy within a horizontal plane.

Thus, the motor power required is reduced virtually to a minimum, since any movement of the drive module along its path and of its drive system occurs in a horizontal plane, without having to fight against gravity.

Height adjustment can even be offered, as this consumes little energy, unlike moving the drive module along its path, which is longer and more frequent. However, it is this movement of the drive module along its path as well as the movement of its drive system which are kept in the horizontal plane, thus allowing optimized reduction of the associated motor power. This embodiment is particularly attractive, as it allows most of the desired movements to occur while minimizing the required motor power and reducing the volume traveled through.

Preferably, the post is not articulated.

The post is thus simpler and more robust.

Preferably, the elongate flexible medical instrument drive system of a robot for insertion of an elongate flexible medical instrument further comprises: an adjustment rail on which the post rests, and a device for locking this post on the adjustment rail during linear movement of the distal end. The adjustment rail is preferably horizontal.

More flexibility is thus provided, allowing more degrees of freedom in the adjustments while retaining the important advantage of greatly reduced motorization or motorizations for the drive module and its drive system.

Advantageously, the adjustment rail rests on an examination table.

Thus, any relative movements between the examination table and the floor no longer need to be compensated for, because they no longer have any impact on the advancement of the elongate flexible medical instrument within the patient.

Advantageously, the elongate flexible medical instrument is a catheter and/or a guide.

A sixth object of the invention relates to a connector between the arterial introducer and the guide tube for the elongate flexible medical instrument of a robot for insertion of an elongate flexible medical instrument. This connector has good functional resistance, in order to properly maintain the elongate flexible medical instrument in the arterial introducer during advancement of the elongate flexible medical instrument within the patient, while ensuring patient safety by eliminating the risk of being torn free during a movement or accidental exertion by the patient that could withdraw the arterial introducer from the patient. This safety is ensured by disconnecting the arterial introducer from the rest of the guide tube for the elongate flexible medical instrument upon patient movement or accidental exertion, this disconnect being caused by said movement or accidental exertion. The connector should pose minimal interference to the elongate flexible medical instrument passing through it, in order not to amplify the slack effect.

To this end, the sixth object of the invention provides a connector between the arterial introducer and the guide tube of the elongate flexible medical instrument of a robot for insertion of an elongate flexible medical instrument, comprising two parts interconnected by at least a first fastener, maintaining the arterial introducer as an extension of the guide tube in order to allow the passage of the elongate flexible medical instrument from the guide tube to the arterial introducer by pushing the elongate flexible medical instrument, the tensile strength along the axis of the elongate flexible medical instrument traversing the connector, of the first fastener prior to releasing one of the two parts of the connector from the other, is less than that of the arterial introducer inserted into the patient before it emerges.

According to preferred embodiments, the invention comprises one or more of the following features which can be used separately or in partial combination or in full combination, not only with the object of the invention mentioned above, but with all objects of the invention mentioned in the rest of this application.

Preferably, the connector between the arterial introducer and the guide tube of the elongate flexible medical instrument of a robot for insertion of an elongate flexible medical instrument, comprises: four parts interconnected by at least a first fastener, maintaining the arterial introducer as an extension of the guide tube to allow the passage of the elongate flexible medical instrument from the guide tube to the arterial introducer by pushing the elongate flexible medical instrument, said first fastener connecting two of the parts located on one side of a transverse plane with the other two parts located on the other side of this transverse plane; comprises at least a second fastener working with the first fastener so that together they immobilize the four parts of the connector, the arterial introducer, and the guide tube, this second fastener connecting two of the parts located on one side of a longitudinal plane with the other two parts located on the other side of this longitudinal plane; the tensile strength, along the axis of the elongate flexible medical instrument traversing the connector, of the first fastener prior to its release, is less than that of the second fastener prior to its release.

Thus, the different elements can easily be inserted into the connector and mounted therein, without these elements needing to have special features enabling quick release, all while enabling smooth passage of the elongate flexible medical instrument from one part to the other of this connector, also, in case of accidental exertion by a patient which could injure the patient, separation occurs quickly and cleanly between these two parts of the connector with each respectively retaining some of the elements.

Preferably, the second fastener works together with a flexible hinge which facilitates the opening and closing of the second fastener.

Preferably, the first fastener comprises at least one central clip, and preferably a plurality of longitudinal clips; the second fastener comprises at least one side clip, and preferably a plurality of side clips.

Fast and clean separation is thus facilitated.

Preferably, the second fastener integrally secures the guide tube to the connector by means of a sleeve surrounding the guide tube, this sleeve being held in place in the connector by the second fastener at several positions along the axis of the sleeve.

The connector can thus easily be adapted to different sizes and/or shapes of arterial introducer, which as a result have different lengths of penetration of the corresponding arterial introducers into the connector, all while more or less continuously guiding the elongate flexible medical instrument between the guide tube and the arterial introducer.

Preferably, the sleeve has ribs along its axis.

The more or less continuous guiding of the elongate flexible medical instrument between the guide tube and the arterial introducer can thus be ensured in a simple manner, admittedly with the use of an additional element but simple in structure.

Preferably, these ribs are arranged periodically along the axis of the sleeve.

Preferably, the number of ribs is between 5 and 15, preferably equal to 10, the dimension of the recess as well as the hump of each rib being between 0.5 mm and 2 mm, preferably equal to 1 mm.

The more or less continuous guiding of the elongate flexible medical instrument between the guide tube and the arterial introducer can thus be provided for a more or less continuous range of values for the penetration of the arterial introducer into the connector.

Preferably, the sleeve is permanently fixed to the guide tube which it surrounds.

The assembly of the connector is thus simplified and shortened, because a basic assembly operation has been eliminated instead of having to be repeated for each new assembly of the connector.

Preferably, the connector comprises a lateral opening enabling the introduction, into the connector, of another tube coming from the arterial introducer.

This new constraint does not interfere with the kinematics of the fast and precipitate disconnect of the connector, nor does it complicate the implementation of the various parts of the connector which enable this quick and precipitate disconnect of the connector in case of accidental and inappropriate exertion by the patient.

Preferably, the connection system comprises: a connector according to any preceding claim, an arterial introducer, and a guide tube for the elongate flexible medical instrument of a robot for insertion of an elongate flexible medical instrument.

Preferably, the connection system comprises a catheter and a guide which are coaxial.

This connection system thus continues to ensure a quick, almost instantaneous disconnect, while allowing the smooth passage not only of a catheter but also a guide.

Preferably, the elongate flexible medical instrument is a catheter and/or a guide.

A seventh object of the invention relates to a set of rotatable rollers arranged so as to work with each other to better guide the elongate flexible medical instrument in translation and in rotation, thus preventing said elongate flexible medical instrument from sliding too much during some of its movements, which otherwise could amplify the slack effect. To improve the guidance of the elongate flexible medical instrument in translation and in rotation, at least three rollers are provided, arranged in a triangle so as to form a deflector for the elongate flexible medical instrument. This triangle of rollers thus acts to deflect the elongate flexible medical instrument. Advantageously, the rollers of this set of rotatable rollers are arranged to work together to better guide the guide as well, in translation and in rotation, and to prevent this guide from sliding too much during some of its movements, which otherwise could amplify the slack effect. To improve the guidance of the guide in translation and in rotation, at least three rollers are provided, arranged in a triangle so as to form a deflector for the guide. This triangle of rollers thus acts to deflect the guide. The improvements to this set of rotatable rollers given in the text below, which are described for the catheter, advantageously can also apply to the guide.

To this end, this seventh object of the invention provides an elongate flexible medical instrument drive module of a robot for insertion of an elongate flexible medical instrument, comprising at least three rotatable rollers, preferably only three rotatable rollers, movable relative to one another so as to come closer together to form a deflector between them during the passage of the elongate flexible medical instrument.

According to preferred embodiments, the invention comprises one or more of the following features which can be used separately or in partial combination or full combination, not only with the object of the invention mentioned above, but with all objects of the invention mentioned in the rest of this application.

Preferably, at least one of the three rollers is a drive motor roller, and preferably only one of the three rollers is a drive motor roller.

Thus, the set of rollers does not need an additional drive element and only requires a limited drive system, because it is then sufficient to drive one of the three rollers.

Preferably, the axes of rotation of the rotatable rollers are parallel to each other and the rollers are circular in a plane perpendicular to their axes of rotation.

The deflector thus remains symmetrical around the elongate flexible medical instrument, thereby preventing the elongate flexible medical instrument from being too compressed on one side and not compressed enough on the other side at some point when traversing the deflector, which provides better guidance of the elongate flexible medical instrument and easier implementation of the deflector.

Preferably, the peripheries of two rollers are tangent to the periphery of a third roller.

The direction of the elongate flexible medical instrument thus remains the same when exiting the deflector as when entering the deflector.

Preferably, the third roller has a larger diameter than that of the other two rollers.

Thus, the length of the elongate flexible medical instrument which is pressing against at least the surface of one roller is increased, which increases the guided length of the elongate flexible medical instrument and which improves the guiding of the elongate flexible medical instrument.

Preferably, the third roller is a roller for driving the elongate flexible medical instrument, while the other two rollers are pressure rollers which press the elongate flexible medical instrument against the driving third roller.

Thus, directly driving only one roller of the three is sufficient, and as the third roller is the largest, it will more easily transmit the driving force to the other two rollers which are smaller.

Preferably, these other two rollers have the same diameter as each other.

Symmetry of the deflector is thus maintained between the entry into the deflector and the exit from the deflector, which further improves the guiding of the elongate flexible medical instrument.

Preferably, in a plane perpendicular to the axes of rotation of the rollers, the created angle whose apex is the center of the third roller and which is formed by the two straight lines respectively connecting the centers of the other two rollers to the center of the third roller, is between 60 degrees and 120 degrees, and is preferably about 90 degrees.

Thus, the amplitude of the changes in direction of the elongate flexible medical instrument as it passes through the deflector remains moderate and is not likely to exert excessive stresses on the elongate flexible medical instrument.

Preferably, at least the third roller and preferably also the other two rollers, has (have) a concave portion for guiding and centering the elongate flexible medical instrument.

Guidance of the elongate flexible medical instrument is thus further enhanced, since the elongate flexible medical instrument remains in the same plane, or at least tends to return to it as soon as it exits therefrom, throughout its passage through the deflector.

Preferably, the concavity of this portion is between one quarter and three quarters of the diameter of the elongate flexible medical instrument, preferably equal to half the diameter of the elongate flexible medical instrument.

This guidance of the elongate flexible medical instrument is thus very effective in keeping the elongate flexible medical instrument centered as it traverses the deflector, and does not exert any excessive stresses which could damage the elongate flexible medical instrument or interfere with its operation.

Preferably, the elongate flexible medical instrument drive system comprises an elongate flexible medical instrument drive module of a robot for insertion of an elongate flexible medical instrument according to the invention, and an elongate flexible medical instrument passing through the deflector formed by the rollers.

Preferably, the elongate flexible medical instrument is a catheter and/or a guide.

An eighth object of the invention relates to a remote control cockpit for a robot for insertion of an elongate flexible medical instrument, which allows the practitioner to have the visibility and time required to guide the elongate flexible medical instrument despite a potential increase in the slack effect, while effectively protecting him or her throughout the manipulation of the elongate flexible medical instrument. Depending on the sizes and morphologies of the practitioner and the patient, and the different sizes and shapes of the devices used, this eighth object of the invention provides a comprehensive system that is modular, in other words a protective shield which is separate from the control station.

For this purpose, this eighth object of the invention provides a remote control cockpit for a robot for insertion of an elongate flexible medical instrument, comprising: a control station for said robot for insertion of an elongate flexible medical instrument which is without an integrated radioprotective shield, and a radioprotective shield that is independent of said control station.

According to preferred embodiments, the invention comprises one or more of the following features which can be used separately or in partial combination or in full combination, not only with the object of the invention mentioned above, but with all objects of the invention mentioned in the rest of this application.

Preferably, said protective shield is movable on the floor, preferably said protective shield rolls on the floor.

The general modularity of the system is thus increased, the decoupling between the relatively motionless if not completely motionless control station and the easily movable protective shield being increased.

Preferably, said control station is movable on the floor, preferably said control station rolls on the floor.

Thus, the general mobility of the protective shield and control station, and their total relative independence, provide optimal modularity of the cockpit.

Preferably, at least a portion of the surface of said protective shield is transparent to visible light, preferably for its entire width and for more than the upper half of its height.

The visibility for the practitioner is thus increased, while remaining sturdy and with a reasonable production cost.

Preferably, said protective shield is a single piece.

The difficult issue of protection at joints and connections in order to reduce or eliminate radiation leaks is thus quite simply avoided.

Preferably, said protective shield comprises at least two planes which are not parallel to each other.

The compromise between effective protection and the dimensions of the protective shield is thus improved.

Preferably, said protective shield comprises some or all of the following: a see-through region transparent to visible light, a region opaque to visible light, wheels with brake, multiple handles arranged to enable one person to roll the protective shield on the floor, preferably means for hanging display screens, for example screens to duplicate angiography images provided with height and/or width adjustment, preferably means for hanging cables.

Preferably, said control station comprises some or all of the following: wheels with brakes, at least one control member, preferably a joystick, at least one monitoring screen, preferably liquid crystal, preferably touchscreen, at least one other human-machine interface, comprising buttons and/or indicator lights which are preferably light-emitting diodes, preferably hooks for accessories, said accessories being for example a remote control for contrast agent injection, a control for an examination table and/or for a C-arm for angiography, a balloon pump.

Preferably, the elongate flexible medical instrument is a catheter and/or a guide.

Other features and advantages of the invention will be apparent from reading the following description of a preferred embodiment of the invention, given by way of example and with reference to the accompanying drawings.

### BRIEF DESCRIPTION OF DRAWINGS

In the following detailed description, the elongate flexible medical instrument is a catheter, but it can also be a guide or some other type of elongate flexible medical instrument.

FIG. 1A schematically represents an exemplary portion of a catheter insertion robot comprising a rail fixed to an arm with a motorized driving module according to one embodiment of the invention.

FIG. 1B schematically represents a front detail view of an exemplary interaction between the rail and the drive module in the insertion robot of FIG. 1A according to one embodiment of the invention.

FIG. 1C schematically represents a side detail view of an exemplary interaction between the rail and the drive module in the insertion robot of FIG. 1A according to one embodiment of the invention.

FIGS. 1D to 1F schematically represent front views of several examples of covers holding the catheter in the rail outside of the area of passage of the drive module, for an insertion robot of FIG. 1A according to one embodiment of the invention.

FIG. 2 schematically represents an exemplary portion of a catheter insertion robot comprising a drive module sliding on a motorized rail supported by an arm according to one embodiment of the invention.

FIGS. 3A and 3B schematically represent a side view and a front view respectively, of a first exemplary sterile barrier in a catheter insertion robot according to one embodiment of the invention.

FIG. 3B′ schematically represents a section view orthogonal to the view of FIG. 3A, of the first exemplary sterile barrier in a catheter insertion robot according to one embodiment of the invention.

FIGS. 3C and 3D schematically represent two front views, respectively outside of the area of passage of the drive module and at the area of passage of the drive module, of a second exemplary sterile barrier in a catheter insertion robot according to one embodiment of the invention.

FIG. 3E schematically represents a side view, at the area of passage of the drive module, of a second exemplary sterile barrier in a catheter insertion robot according to one embodiment of the invention.

FIGS. 3F and 3G schematically represent a perspective view and a front view respectively, of a third exemplary sterile barrier in a catheter insertion robot according to one embodiment of the invention.

FIG. 3H schematically represents a side view of a fourth exemplary sterile barrier in a catheter insertion robot according to one embodiment of the invention.

FIG. 3I schematically represents a side view of a fifth exemplary sterile barrier in a catheter insertion robot according to one embodiment of the invention.

FIG. 3J schematically represents a side view of a sixth exemplary sterile barrier in a catheter insertion robot according to one embodiment of the invention.

FIG. 3K schematically represents a side view of a seventh exemplary sterile barrier in a catheter insertion robot according to one embodiment of the invention.

FIG. 4A schematically represents a perspective view of a first exemplary catheter guide track in a catheter insertion robot according to one embodiment of the invention.

FIGS. 4B-4C schematically represent respective front views of two alternatives of the first exemplary catheter guide track in a catheter insertion robot according to one embodiment of the invention.

FIG. 4D schematically represents a perspective view of a second exemplary catheter guide track in a catheter insertion robot according to one embodiment of the invention.

FIG. 4E schematically represents a perspective view of a third exemplary catheter guide track in a catheter insertion robot according to one embodiment of the invention.

FIG. 4F schematically represents a perspective view of a fourth exemplary catheter guide track in a catheter insertion robot according to one embodiment of the invention.

FIGS. 4G and 4H schematically represent respective front views of two positions, open and closed, of the fourth exemplary catheter guide track in a catheter insertion robot according to one embodiment of the invention.

FIG. 4I schematically represents a perspective view of a fifth exemplary catheter guide track in a catheter insertion robot according to one embodiment of the invention.

FIG. 4J schematically represents a perspective view of a sixth exemplary catheter guide track in a catheter insertion robot according to one embodiment of the invention.

FIG. 4K schematically represents a perspective view of a seventh exemplary catheter guide track in a catheter insertion robot according to one embodiment of the invention.

FIG. 4L schematically represents a perspective view of an eighth exemplary catheter guide track in a catheter insertion robot according to one embodiment of the invention.

FIG. 4M schematically represents a perspective view of a ninth exemplary catheter guide track in a catheter insertion robot according to one embodiment of the invention.

FIG. 5A schematically represents a side view of a first exemplary arm supporting a catheter drive module in a catheter insertion robot according to one embodiment of the invention.

FIG. 5B schematically represents a side view of a second exemplary arm supporting a catheter drive module in a catheter insertion robot according to one embodiment of the invention.

FIG. 5C schematically represents a side view of a third exemplary arm supporting a catheter drive module in a catheter insertion robot according to one embodiment of the invention.

FIG. 5D schematically represents a side view of a fourth exemplary arm supporting a catheter drive module in a catheter insertion robot according to one embodiment of the invention.

FIG. 5E schematically represents a side view of a fifth exemplary arm supporting a catheter drive module in a catheter insertion robot according to one embodiment of the invention.

FIGS. 5F and 5G schematically represent respective perspective views of a sixth exemplary arm supporting a catheter drive module in a catheter insertion robot according to one embodiment of the invention.

FIGS. 6A and 6B schematically represent perspective views, respectively in an assembled position and in a separated position, of an exemplary arterial introducer connector in a catheter insertion robot according to one embodiment of the invention.

FIGS. 7A and 7B schematically represent top views of an exemplary set of guide rollers of a catheter drive module in a catheter insertion robot according to one embodiment of the invention.

FIG. 7C schematically represents a side view of an exemplary drive roller in a set of guide rollers of a catheter drive module in a catheter insertion robot according to one embodiment of the invention.

FIG. 8A schematically represents a side view of an exemplary remote control station for a catheter drive module in a catheter insertion robot, incorporating a radioprotective shield, according to one embodiment of the invention.

FIG. 8B schematically represents a perspective view of another exemplary remote control station for a catheter drive module in a catheter insertion robot, incorporating a radioprotective shield, according to one embodiment of the invention.

### LIST OF REFERENCES IN THE FIGS

1/catheter
2/guide
3/drive module
4/rail of drive module
5/arm (robotic)
6/sterile barrier
7/table
8/arterial introducer (or desilet)
9/patient
10/adjustment rail
11/post
12/ball joint
13/fixed attachment
14/motor portion of the drive module
15/support portion of the drive module
16/power cord
17/protrusions from the support portion of the drive module
18/grooves of the rail of the drive module
19/groove cover
30/wrinkled portion
31/retaining elastic member
32/fold
33/skirt for drive module
34/winder
35/attachment point
36/cowl
37/glued skirt
38/weight
39/support post of drive module
40/guide track
41/chain
42/flap
43/slit tube
44/connection
45/zipper
46/spiral
48/winder
49/band
50/segment
51/joint
52/support post
53/anchorage
54/adjustment arm
60/connector
61/guide tube
62/connector part
63/central clip
64/side clip
65/sleeve
66/rib
67/lateral opening
68/“luer-lock” type female fitting
69/hinge
70/deflector
71/drive roller
72/periphery of drive roller
73/pressure roller
74/periphery of pressure roller
75/guide portion
80/shield
81/see-through portion of shield
82/opaque portion of shield
83/wheeled support
84/control station
85/chair
86/physician
87/lateral hose connected to the arterial introducer
88/longitudinal openings of the connector
100/spreading front portion of the drive module
101/spreading rear portion of the drive module
102/floor
160/handles of sterile barrier
180/rollers
190/cap
191/clip
192/guide tab
400/section
401/bottom
402/side member
403/female closure
404/male closure
405/thinned area
406/beveled edge
407/wide cavity
410/notched half chain
411/notches
412/bellows
413/fold
414/overhang
801/viewing screens
802/grip handles
803/wheels
804/wheel brakes
805/first plate, plane of protective shield
806/second plate, plane of protective shield
840 wheeled support
841/wheels
842/wheel brakes
843/body of control station
844/work desk of control station
845/monitoring screen
846/joysticks
847/accessory hooks

### DETAILED DESCRIPTION OF INVENTION

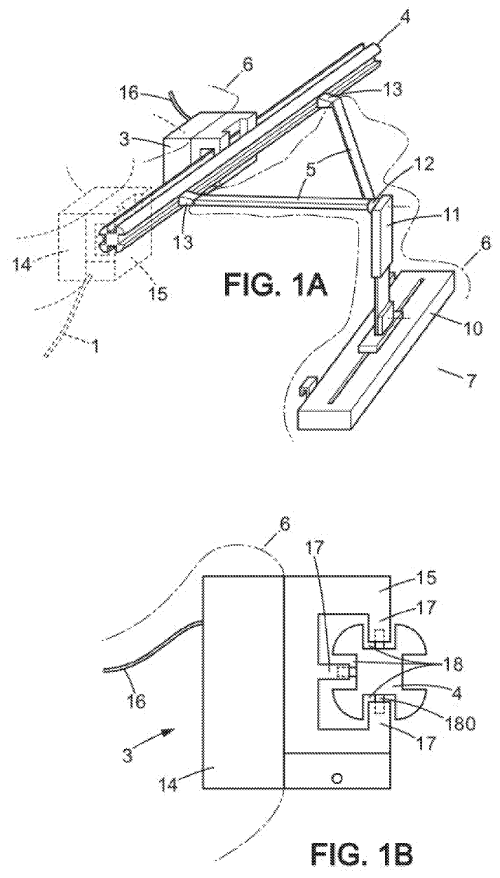

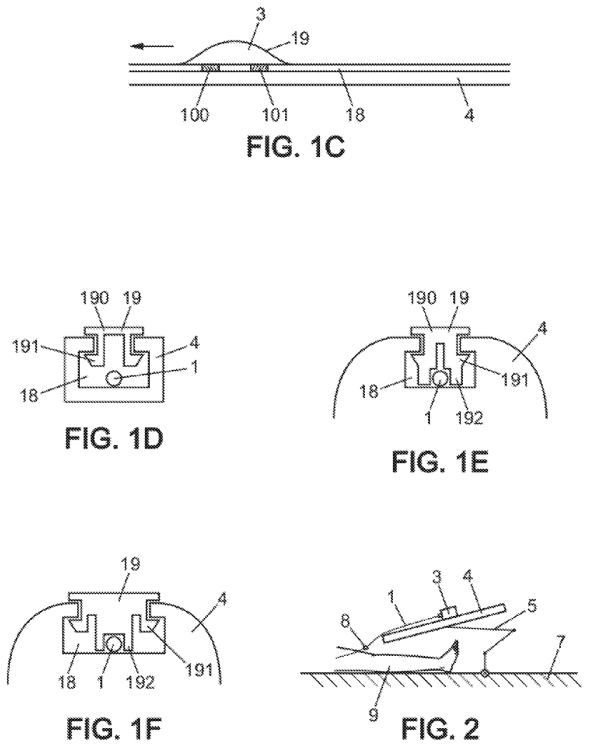

FIG. 1A schematically represents an exemplary portion of a catheter insertion robot comprising a rail fixed to an arm with a motorized driving module according to one embodiment of the invention.

An adjustment rail 10 is fixed to a table 7. A post 11 moves horizontally on this rail 10. This vertical post 11 is secured to the adjustment rail 10 by its lower end and is vertically extensible. An arm 5 with two branches is secured to the upper end of the post 11 by means of a ball joint 12. Each branch of the arm 5 is fixed to a rail 4 of the drive module 3 by a fixed attachment 13. A drive module 3 moves along this rail 4 by sliding on the rail 4. The drive module 3 is composed of two portions, a motor portion 14 of the drive module 3 and a support portion 15 of the drive module 3. It is the support portion 15 of the drive module 3 which is in contact with the rail 4 and which slides on the rail 4 when the drive module 3 slides on the rail 4. The motor portion 14 of the drive module 3 is supplied power by a power cord 16. A catheter 1 is connected or attached to the support portion 15 of the drive module 3.

A first sterile barrier 6 surrounds the adjustment rail 10, post 11, and arm 5. A second sterile barrier 6 surrounds the motor portion 14 of the drive module 3, passing between the motor portion 14 and the support portion 15 of the module drive 3. These two sterile barriers 6 surround the reusable components. Between these two sterile barriers 6 are the rail 4 with its fixed attachments 13, as well as the support portion 15 of the drive module 3 and the catheter 1.

First, the post 11 slides on the adjustment rail 10 until it reaches the desired position, determined by the patient's position on the table 7 as well as the position of the corresponding arterial introducer at the point of entry of the catheter 1 into the patient. Once this adjustment position of the post 11 on the adjustment rail 10 is found, this adjustment position is locked and the post 11 becomes stationary in translation on the adjustment rail 10. Next, the post 11 is extended vertically to the proper height, for example by telescoping. Once the proper height of the post 11 is obtained, this height is locked. Lastly, the ball joint 12 is used to obtain the desired orientation for the arm 5 carrying the rail 4 and the drive module 3. Once this orientation is obtained, it is locked to immobilize this suitable orientation for entry of the catheter 1 into the arterial introducer already in place in the patient. The rail 4 is fixed to the arm 5 by the fixed attachments 13. Finally, the drive module 3 slides on the rail 4, moving from the extreme position indicated in dashed lines in FIG. 1A to a central position indicated in solid lines, drawing the catheter 1 and inserting it into the patient through the arterial introducer.

The rail 4 is a section of disposable aluminum. To reduce the footprint of the rail 4, it could be delivered in pieces and then assembled.

This would also simplify its disposal.

The motor portion 14 of the rail 4 is a reusable motorized carriage. It houses the entire drive module 3. The drive module 3 receives the catheter 1 and the guide 2. The catheter 1 is advanced by movement of the motor portion 14 along the rail 4, while rotation is achieved by a mechanism which is part of the drive module 3. The guide 2 is controlled, in translation and in rotation, by means of the drive module 3.

The post 11 could also tilt in order to bring the end of the rail 4 close to the patient and therefore reduce the dead length. A worm screw system would be used for this. A handcrank would then be installed at the end of the screw to facilitate its use. To adjust the height in order to adapt to different patient morphologies, the post 11 is for example telescoping, and actuated by a handcrank placed on its lower portion. Its movement is then ensured by a worm screw system coupled to a guide shaft.

The linear rail 10 allows pre-positioning the assembly prior to beginning the operation. It is equipped with two handles, one having a button. This button will be used to release a brake or catch system in order to move the entire arm 5 manually relative to the linear rail 10. The linear rail 10 is separate from and independent of the rail DIN of the table 7.

FIG. 1B schematically represents a front detail view of an exemplary interaction between the rail and the drive module in the insertion robot of FIG. 1A according to one embodiment of the invention.

The second sterility barrier 6 passes between the motor portion 14 and the support portion 15 of the drive module 3. The drive module 3 is hooked to the rail 4 by means of its support portion 15. The support portion 15 of the drive module 3 comprises three projections 17 which give an E shape to the inside of this support portion 15. These three projections 17 enter three grooves 18 of the rail 4 and engage with the three grooves 18 to slide within these three grooves 18, for example by rolling on rollers 180 at the bottom of these grooves 18. These rollers 180 are preferably mounted on the consumable portion 15 of the drive module 3.

The support portion 15 of the drive module 3 has an E shape in order to slide in three of the grooves 18 of the rail 4 while being driven by rollers 180. Several shoes, some of them presser shoes, may be added to keep the assembly in position. The grooves 18 of the rail 4 could also serve as tracks for the catheter 1 and/or a guide 2. In this case, a cover 19 is preferably installed as shown in FIGS. 1C to 1F.

FIG. 1C schematically represents a side detail view of an exemplary interaction between rail and drive module in the insertion robot of FIG. 1A according to one embodiment of the invention.

The direction of movement of the drive module 3 is indicated by the arrow. When moving the drive module 3 along the rail 4, a spreading front portion 100 of the drive module 3 raises the cover 19 covering the groove 18 of the rail 4 to allow the drive module 3 to pass, and a rear portion 101 lowers the cover 19 so that the groove 18 of the rail 4 is once again covered after the passage of the drive module 3. In this manner, the cover 19 is only lifted or moved away, depending on the embodiments, at the moment of passage of the drive module 3, which reduces the risk of dust entering the groove 18 of the rail 4 where the catheter 1 travels.

FIGS. 1D to 1F schematically represent front views of several exemplary covers keeping the catheter in the rail, outside the area of passage of the drive module, for an insertion robot of FIG. 1A according to one embodiment of the invention.

In FIG. 1D, in a first embodiment, the catheter 1 advances at the bottom of a groove 18 of the rail 4, driven by the displacement of the drive module 3. A cover 19 covers the catheter 1 in the groove 18. This cover 19 comprises a cap 190 which is extended on each side by a clip 191 that enables fitting the cover 19 into the groove 18 to form a protective channel for the catheter 1.

In FIG. 1E, in a second embodiment, the catheter 1 advances at the bottom of a groove 18 of the rail 4, driven by the displacement of the drive module 3. A cover 19 covers the catheter 1 in the groove 18. This cover 19 comprises a cap 190 which is extended on each side by a clip 191 that enables fitting the cover 19 into the groove 18 to form a protective channel for the catheter 1. Each clip 191 is extended by a guide tab 192, the catheter 1 being held between the two guide tabs 192.

In FIG. 1F, in a third embodiment, the catheter 1 advances at the bottom of a groove 18 of the rail 4, driven by the displacement of the drive module 3. A cover 19 covers the catheter 1 in the groove 18. This cover 19 comprises a cap 190 extended on each side by a clip 191, at each end of the cap 190, which enables fitting the cover 19 into the groove 18 to form a protective channel for the catheter 1. The cap 190 is extended on each side by a guide tab 192 at each median portion of the cap 190, so that the catheter 1 can be held between the two guide tabs 192.

FIG. 2 schematically represents an exemplary portion of a catheter insertion robot comprising a drive module sliding on a motorized rail supported by an arm according to one embodiment of the invention.

A patient 9 is lying on a table 7. An arm 5 is arranged on the table 7. An arterial introducer 8 has been placed in a limb of the patient 9, for example a leg or arm of the patient 9. A catheter 1 is gradually inserted into the arterial introducer 8. The arm 5 carries the rail 4. The drive module 3 causes insertion of the catheter 1 into the arterial introducer 8, by sliding on the rail 4. Motorization is ensured by the rail 4 which is motorized and which drives the drive module 3 which itself is not motorized.

The rail 4 is carried by a mechanical arm 5 connected to the table 7. This mechanical arm 5 has several degrees of freedom for bringing the end of the rail 4 next to the patient 9.

Next, the drive module takes charge of the catheter 1 and guide 2. It handles the rotation of the catheter 1 and the translation and rotation of the guide 2, the translation of the catheter 1 being achieved by the movement of the drive module 3 along the rail 4.

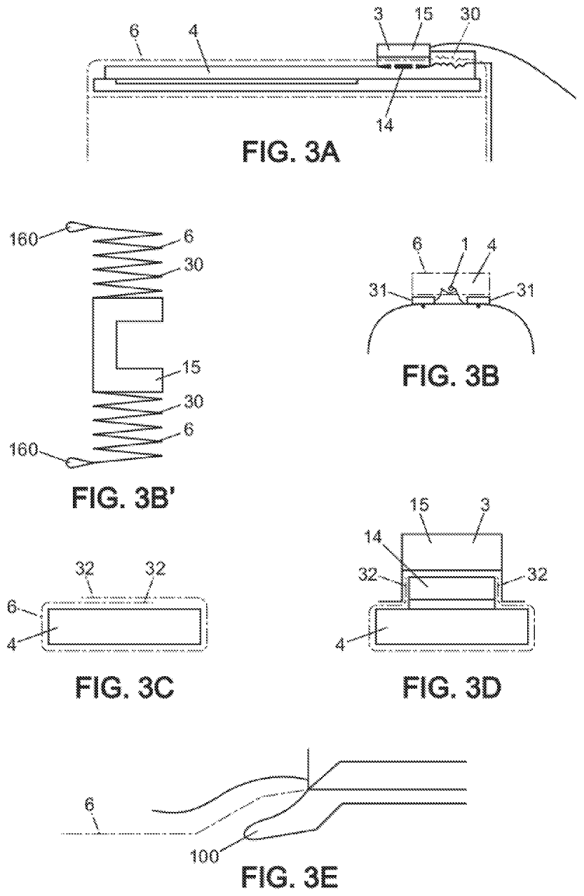

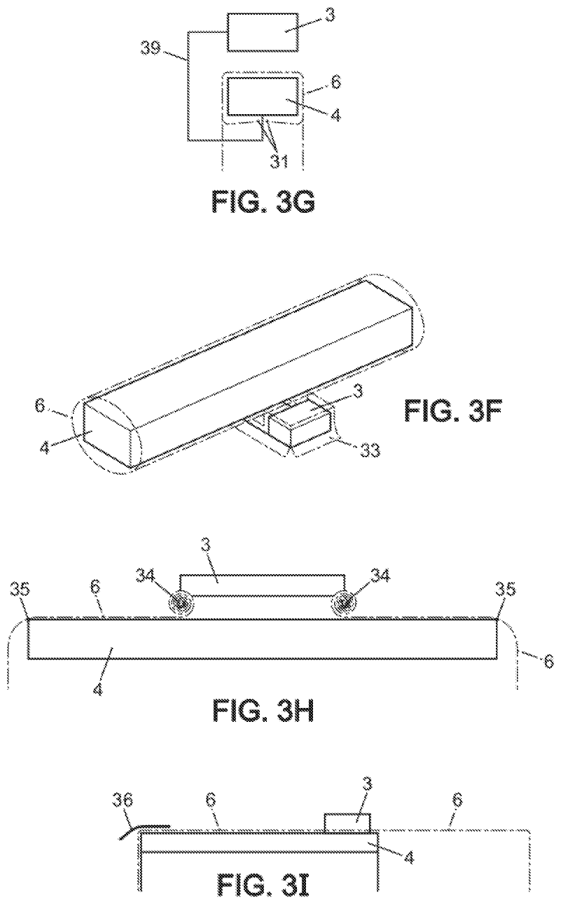

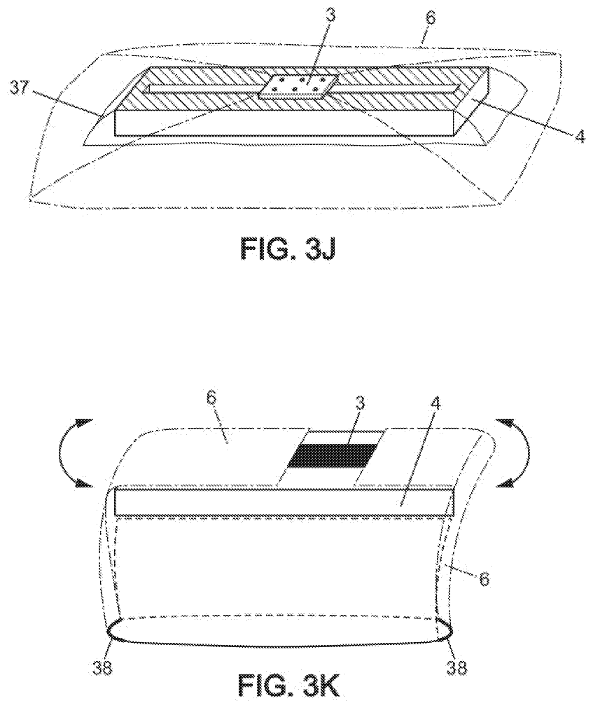

FIGS. 3A and 3B schematically represent a respective side view and front view of a first exemplary sterile barrier in a catheter insertion robot according to one embodiment of the invention.

In the text, unless otherwise stated, the terms sterile skirt and sterile barrier are used interchangeably.

In FIG. 3A, the drive module 3 can slide from one side of the rail 4 to the other, and vice versa. The sterile barrier 6 extends over the entire length of the rail 4, passing through the middle of the drive module 3 between the motor portion 14 and the support portion 15. Here, the motor portion 14 is by the rail 4 which is motorized and which drives the motor portion 14. Here, the actual motor is more on the rail 4 side than on the drive module 3 side. The motor portion 14 is reusable, while the support portion 15 is disposable. On each side of the drive module 3, the sterile barrier 6 comprises a wrinkled portion 30 which can be stretched or compressed depending on whether the movement of the drive module is in one direction or the other. One of the wrinkled portions 30 is compressed when the drive module 3 pushes it and the other wrinkled portion 30 simultaneously stretches because the drive module 3 is pulling it, and then vice versa.

In FIG. 3B, the sterile barrier 6 comes to cover the rail 4 from below, and holds the wrinkled portions 30 around the rail 4 by means of retaining elastic members 31 arranged on each side transversely to the direction of the rail 4.

FIG. 3B′ schematically represents a side view, orthogonal to the view of FIG. 3A, of the first exemplary sterile barrier in a catheter insertion robot according to one embodiment of the invention. The sterile barrier 6 is shown by itself with the support portion 15 of the drive module 3, in a folded configuration. On each side, longitudinally relative to the rail 4 (not shown), is the wrinkled portion 30 of the sterile barrier 6 in the folded configuration. At the end of each wrinkled portion 30 that is opposite to the support portion 15, there is a handle 160 which when pulled helps to unfold the sterile barrier 6 and install it around the rail 4.

The skirt 6 is wrinkled on both sides of the drive module 3, and to maintain these wrinkled portions 30 and prevent the skirt 6 from touching the catheter 1 or any other external element, elastic members 31 have been added on each side the rail 4. The wrinkled portions 30 allow absorbing the translations of the drive module 3 along the rail 4.

FIGS. 3C and 3D schematically represent two front views, respectively outside the area of passage of the drive module and within the area of passage of the drive module, of a second exemplary sterile barrier in a catheter insertion robot according to one embodiment of the invention.

In FIG. 3C, the rail 4 is surrounded by the sterile barrier 6, two folds 32 overlapping one another to cover the upper portion of the rail 4. FIG. 3C shows the configuration of the sterile barrier 6 surrounding the rail 4, on the portions of the rail 4 where the drive module 3 is not present.

In FIG. 3D, the rail 4 is surrounded by the sterile barrier 6, two folds 32 lifting to face one another so that the upper portion of the rail 4 is briefly uncovered and allowing the passage of the drive module 3. FIG. 3D shows the configuration of the sterile barrier 6 where it only partially surrounds the rail 4, on the portion of the rail 4 when the drive module 3 is present.

The split skirt 6 opens at the passage of the drive module 3, and once it is open, its “channel” design maintains the sterility.

FIG. 3E schematically represents a side view, at the area of passage of the drive module, of a second exemplary sterile barrier in a catheter insertion robot according to one embodiment of the invention.

The spreading front portion 100 of the drive module 3, as it passes, moves apart the two folds 32 that were in the folded configuration of FIG. 3C in order to place them in the unfolded configuration of FIG. 3D, before the spreading rear portion 101 of the drive module 3 (not shown here) returns them to the folded configuration of FIG. 3C.

FIGS. 3F and 3G schematically represent a perspective view and a front view, respectively, of a third exemplary sterile barrier in a catheter insertion robot according to one embodiment of the invention.

In FIG. 3F, a first sterile barrier 6 surrounds the rail 4, the sterile barrier 6 being stationary relative to the rail 4, while a second sterile barrier 33 surrounds the drive module 3. The first sterile barrier 6 is stationary relative to the rail 4. The drive module 3 slides under the rail 4 to which it is hooked. A slot in the sterile barrier 6 allows the passage for example of a support post of the drive module 3, while reducing the risk of incoming contamination.

In FIG. 3G, the retaining elastic members 31 for the first sterile barrier 6 surrounding the rail 4 are represented. As the drive module 3 slides below the rail 4, the first sterile barrier 6 opens between the two retaining elastic members 31 to allow the passage of the drive module 3 and then closes after it has passed.

Preferably, the rail 4 which is in fact in the reversed position, i.e. with its movable portion at the bottom, comprises a support post 39 on which the drive module 3 is fixed. The drive module 3 and the support post will be covered with a blister 33 which closely fits the shape of the drive module 3 and its support post 39. The rail 4 is provided with a protective cover 6 having an elastic member 31 which will tighten around it.

FIG. 3H schematically represents a side view of a fourth exemplary sterile barrier in a catheter insertion robot according to one embodiment of the invention.

The drive module 3 comprises two winders 34, one on each longitudinal side relative to the drive module 3, one at the front and the other at the rear. The sterile barrier 6, which surrounds the rail 4, is fixed to the rail 4 at two attachment points 35 located at the two ends longitudinally along the rail 4. When the drive module 3 moves to one side, the winder 34 located at the front of the drive module 3 relative to its movement, “swallows” the sterile barrier 6 as the drive module 3 advances, until it reaches one of the attachments points 35, while the other winder 34 which is located at the rear of the drive module 3 relative to its movement, “unwinds” the sterile barrier 6 as the drive module 3 moves away from the other attachment point 35. When the drive module 3 starts moving again to the other side, the winder 34 located in the front of the drive module 3 relative to its movement, “swallows” the sterile barrier 6 as the drive module 3 moves, until reaching one of the attachment points 35, while the other winder 34 which is located at the rear of the drive module 3 relative to its movement, “unwinds” the sterile barrier 6 it had swallowed in the previous step, as the drive module 3 moves away from the other attachment point 35.

FIG. 3I schematically represents a side view of a fifth exemplary sterile barrier in a catheter insertion robot according to one embodiment of the invention.

The sterile barrier 6 is smooth and is a very long, typically about twice the length of the rail 4. At one of the longitudinal ends of the rail 4 there is a cowl 36 curving downward and located above the rail 4. The sterile barrier 6, when pushed by the drive module 3 towards the cowl 36, will be directed downwards by the cowl 36 and therefore will slide between the cowl 36 and the rail 4. Conversely, when the drive module 3 moves in the other direction, a significant excess in the length of the sterile barrier 6 hangs beyond the rail 4.

The skirt 6 is long enough to absorb the movements of the drive module 3, preferably at least twice the path length of the rail 4. For safety reasons, a cowl 36 is placed on the side closest to the arterial introducer (or desilet) to direct the skirt 6 downwards and thus away from the arterial introducer. In this configuration, the drive module may be positioned on top of the rail 4, or below the rail 4, or to the side of the rail 4.

FIG. 3J schematically represents a view of a sixth exemplary sterile barrier in a catheter insertion robot according to one embodiment of the invention.

In addition to the sterile barrier 6 covering the rail 4 and traversing the drive module 3, thereby separating the support 15 and motor 14 portions of the drive module 3, another sterile skirt 37 located below the sterile barrier 6 covers closer to the rail 4, being placed on the rail 4, preferably glued to the rail 4, without including any portion of the drive module 3. This dual sterile protection improves the level of protection, although it requires using two sterile skirts of different sizes and shapes.

FIG. 3K schematically represents a view of a seventh exemplary sterile barrier in a catheter insertion robot according to one embodiment of the invention.

The sterile barrier 6 extends completely around the rail 4, but transversely, meaning about an axis perpendicular to the longitudinal axis of the rail 4, and not about the longitudinal axis of the rail 4 as in other embodiments. This sterile barrier 6 comprises weights 38, to prevent the free portion of the sterile barrier 6, which is not around the rail 4, from interfering with the rest of the device. As the drive module 3 moves, the sterile barrier 6 rotates about an axis perpendicular to the longitudinal axis of the rail 4, in the direction indicated by the arrows in FIG. 3K.

The skirt 6 is therefore rotated about the rail 4 when the drive module 3 moves. The weights 38 in the skirt 6 ensure that it hangs properly and does not interfere with the mechanics located above it. Another solution would be to add guide flanges on the sides where they do not interfere with the external elements, while ensuring that these flanges are sealed.

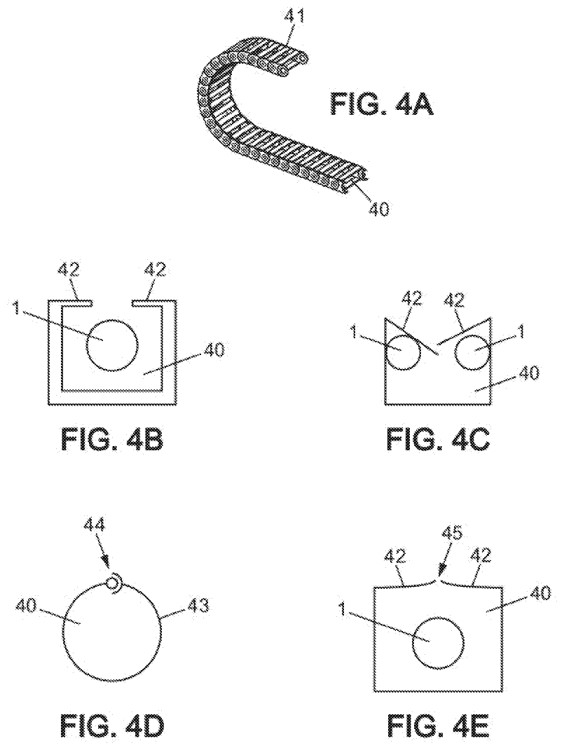

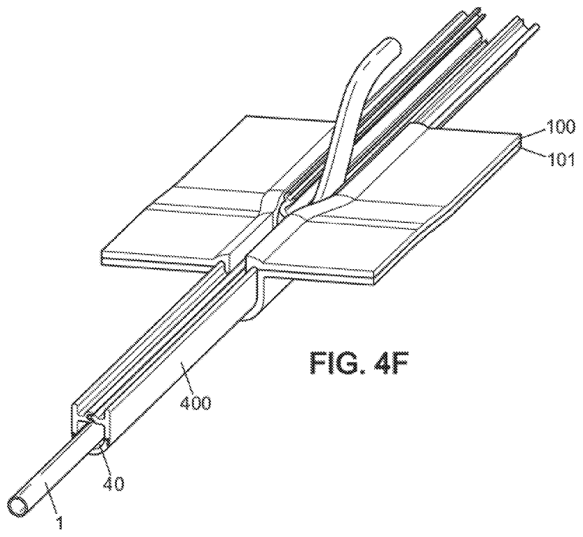

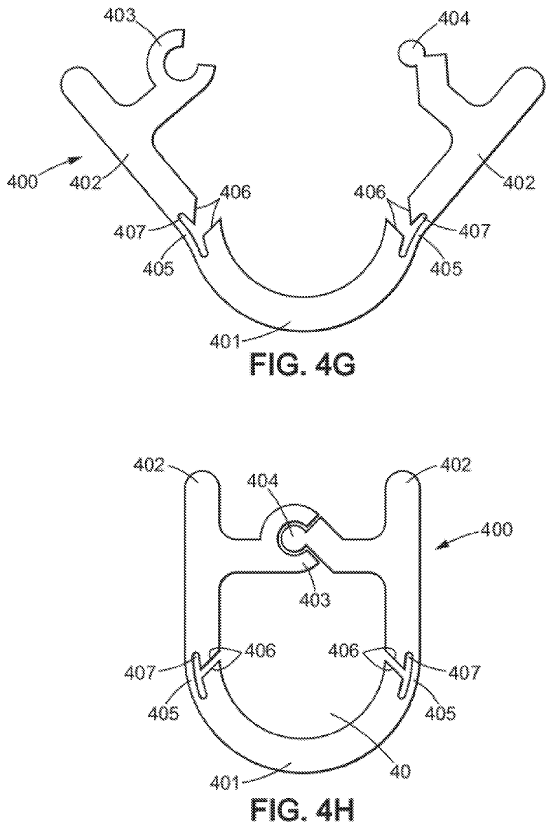

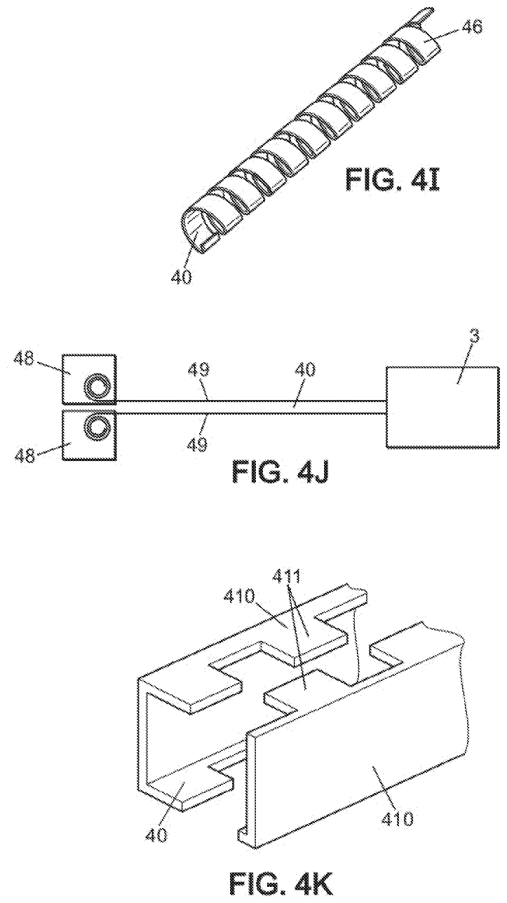

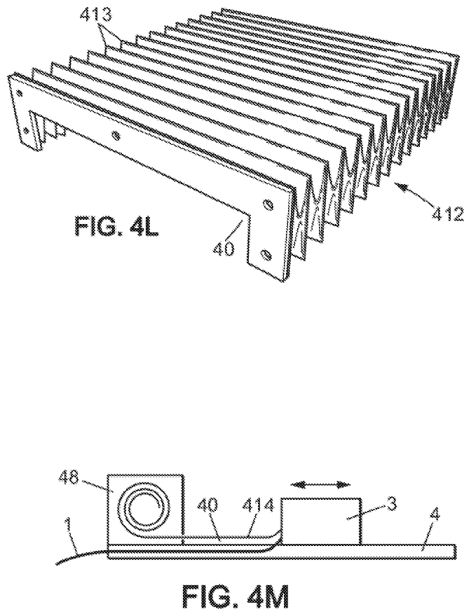

FIG. 4A schematically represents a perspective view of a first exemplary catheter guide track in a catheter insertion robot according to one embodiment of the invention.

A chain 41, such as a “drag chain”, defines a guide track 40 in its structure, within which a catheter 1 and guide 2 represented only in FIGS. 4B and 4C will be guided.

FIGS. 4B-4C schematically represent respective front views of two alternatives of the first exemplary catheter guide track in a catheter insertion robot according to one embodiment of the invention. This catheter guide track can also be used to guide a guide.

In the first example of FIG. 4B, a catheter 1 is inserted into the guide track 40, through the space between the flexible flaps 42. This space is smaller than the diameter of the catheter 1 which pushes the flaps 42 inward during its insertion into the guide track 40, these flaps 42 elastically and automatically resuming their horizontal position after the passage of the catheter 1.

In the second example of FIG. 4C, two possible positions of the catheter 1 are shown relative to the guide track 40. The catheter 1 is inserted into the guide track 40 through the space between the flexible flaps 42. This space is smaller than the diameter of the catheter 1, the latter pushing the flaps 42 inward during its insertion into the guide track 40, the flaps 42 elastically and automatically resuming their oblique position after the passage of the catheter 1.

FIG. 4D schematically represents a perspective view of a second exemplary catheter guide track in a catheter insertion robot according to one embodiment of the invention.

The guide track 40 consists of the interior of a slotted tube 43, which is opened to allow a catheter 1 to pass inside and which is closed after the passage of the catheter 1, at a connection 44.

FIG. 4E schematically represents a perspective view of a third exemplary catheter guide track in a catheter insertion robot according to one embodiment of the invention.

The guide track 40 is a channel that will accommodate a catheter 1 after it passes through a zipper closure 45 joining two horizontal flaps 42. When introducing the catheter 1 into the guide track 40, the zipper closure 45 is opened to allow the catheter 1 to pass inside, and then is closed again after the passage of the catheter 1.

FIG. 4F schematically represents a perspective view of a fourth exemplary catheter guide track in a catheter insertion robot according to one embodiment of the invention.

The catheter 1 is guided in a section 400, which is opened to allow the catheter 1 to pass inside the section 400 and which is closed after the passage of the catheter 1 in order to guide it inside the section 400. This section 400 is opened by a front portion 100 of the drive module 3 and is closed again by a rear portion of the drive module 3.

FIGS. 4G and 4H schematically represent respective front views of two positions, open and closed, of the fourth exemplary catheter guide track in a catheter insertion robot according to one embodiment of the invention.

In FIG. 4G, the section 400 is in the open position to allow a catheter 1 to pass inside when inserted into the section 400. The section 400 comprises a bottom 401, two side members 402, a female closure 403, a male closure 404, two thinned areas 405, four beveled edges 406, and two wide cavities 407. The beveled edges 406 are divided into two pairs, one on each side of the bottom 401, and for each pair, one of the beveled edges 406 is on the side member 402 while the other is on the bottom 401. Between the beveled edges 406 of each pair and the corresponding thinned area 405 there is an enlarged cavity 407 further improving the flexibility of the thinned area 405 while making it more robust because, since the surface area of the thinned area 405 is increased, the force to be exerted on it will be better distributed.

The bottom 401, which is rounded, is respectively connected at its two ends to the side members 402, respectively via the thinned areas 405. One of the side members 402 carries the male closure 404, while the other side member 402 carries the female closure 403.

The male closure 404 is out of the female closure 403. Their respective side members 402 have been moved apart from one another by deforming their thinned areas 405 which are inherently more flexible than the other areas of the section 400, due to their thinness. The beveled edges 406 of each pair have also moved apart from one another.

In FIG. 4H, the section 400 is in the closed position for guiding a catheter 1.

The male closure 404 is inserted into the female closure 403. Their respective side members 402 have moved toward one another and are once again parallel to constitute the side walls of the guide track 40. The beveled edges 406 of each pair have also moved toward one another, until they are almost in contact with one another. The guide track 40 now forms a channel having an almost continuous inner wall.

FIG. 4I schematically represents a perspective view of a fifth exemplary catheter guide track in a catheter insertion robot according to one embodiment of the invention.

The guide track 40 is a spiral 46, the catheter 1 being inserted between its turns in order to be guided and moved by the rotation of the spiral 46 about its own axis of symmetry.

FIG. 4J schematically represents a perspective view of a sixth exemplary catheter guide track for a catheter insertion robot according to one embodiment of the invention.

The guide track 40 is located between bands 49 which wind and unwind from winders 48. When the drive module 3 moves towards the winders 48, pushing a catheter 1 (not shown) in the guide track 40 as the drive module 3 advances towards an arterial introducer, the bands 49 are wound on the winders 48.

The two winders 48, which are placed close to the arterial introducer, each contain a portion of the zipper system. Exiting from the winders 48, the two bands 49, which are the two portions of the zipper, are joined to form a single wide band. The end of this wide band is integrally secured to the drive module 3.

FIG. 4K schematically represents a perspective view of a seventh exemplary catheter guide track in a catheter insertion robot according to one embodiment of the invention.

The guide track 40 is the interior of a drag chain consisting of the assembly of two half-chains 410 having respective complementary slots 411 which fit into one another.

The drag chain is formed of two flexible portions which are the half chains 410 and which, when the half-chains 410 are joined together by fitting one into the other, are stiffened and hold the catheter 1 in place.

FIG. 4L schematically represents a perspective view of an eighth exemplary catheter guide track for a catheter insertion robot according to one embodiment of the invention.

The guide track 40 is inside a bellows 412 comprising a number of pleats 413. This bellows 412 can compress or extend depending on the direction of movement of the drive module 3. This bellows 412 covers the guide track 40 from above.

FIG. 4M schematically represents a perspective view of a ninth exemplary catheter guide track in a catheter insertion robot according to one embodiment of the invention.

The guide track 40 for the catheter 1 is composed of a channel covered by an overhang 414. Depending on the direction of movement of the drive module 3, the overhang 414 winds into the winder 48 or unwinds from the winder 48, so as to cover the portion of the guide track 40 located between the drive module 3 and the arterial introducer.

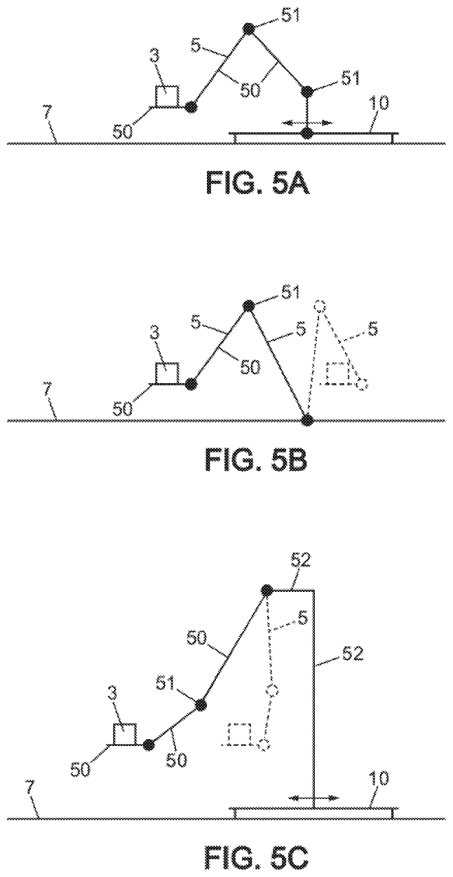

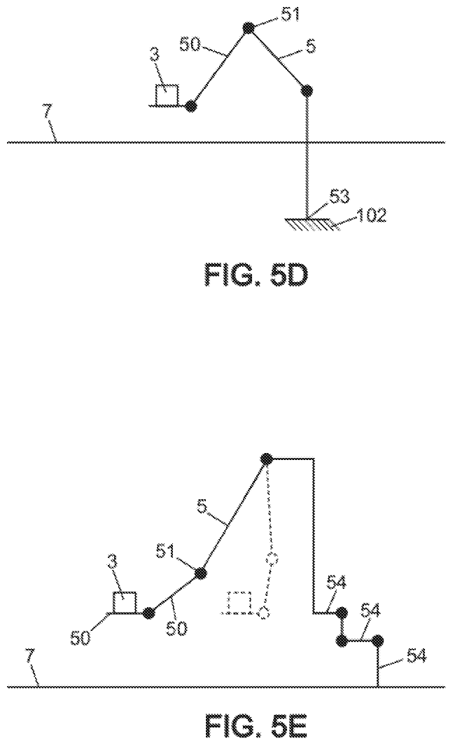

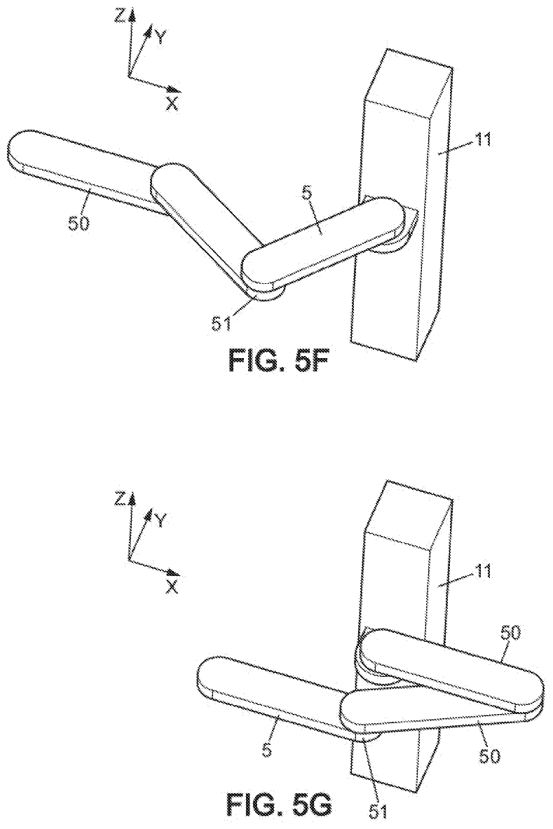

FIG. 5A schematically represents a side view of a first exemplary arm supporting a catheter drive module in a catheter insertion robot according to one embodiment of the invention.

The catheter drive system of a catheter insertion robot comprises an articulated arm 5 comprising at least three segments 50, here four segments 50, pivotally connected by joints 51, advantageously ball joints or at least pivot joints. This robotic arm 5 is adapted to trace a linear path through space at its distal end which carries a catheter drive module 3 integrally secured to the distal end.

In order, from patient to table 7, we find the drive module 3 carried by a first segment 50 at the distal end of the arm 5, a second segment 50, a third segment 50, and a fourth segment 50 connected at its proximal end to an adjustment rail 10. The distal end of the arm 5 is its end closer to the patient while the proximal end of the arm 5 is its end closer to the table 7.

Once the proximal end of the arm 5 is immobilized along the adjustment rail 10 relative to the size and position of the patient as well as relative to the arrangement of the arterial introducer in the patient, the various segments 50 pivot so as to send the drive module 3 along a linear path that will insert the catheter into the arterial introducer by means of linear movement of the drive module 3.

The adjustment phase is carried out by moving the entire arm 5 along the adjustment rail 10, while the movement phase of the drive module 3 is carried out by deployment of the arm 5, its segments 50 pivoting on joints 51.

As the robotic arm 5 is mounted on an adjustment rail 10 which in turn is fixed to the edge of the table 7, the drive system is adjusted relative to the end point by means of the adjustment rail 10, manually by the user, then the selected position is locked in place. As the position of the robotic arm 5 is then fixed, only the portion of the path corresponding to the advancement of the catheter 1 into the patient needs to be managed.

FIG. 5B schematically represents a side view of a second exemplary arm supporting a catheter drive module in a catheter insertion robot according to one embodiment of the invention.

The catheter drive system of a catheter insertion robot comprises an articulated arm 5 comprising three segments 50 pivotally connected together by joints 51 and robotized, able to trace a linear path through space at its distal end which carries a catheter drive module 3 integrally secured to this distal end.

In order, from patient to table 7, we find the drive module 3 carried by a first segment 50 at the distal end of the arm 5, a second segment 50, and a third segment 50 directly connected at its proximal end to the table 7 by a ball joint.

The various segments 50 pivot to cause the drive module 3 to trace a linear path in order to insert the catheter into the arterial introducer by means of linear movement of the drive module 3.

The adjustment phase and the movement phase of the drive module 3 are carried out simultaneously by deployment of the arm 5, its segments 50 pivoting on the joints 51. Alternatively, a portion of the arm 5, nearer its proximal end, can first carry out the adjustment phase, while another portion of the arm 5, nearer its distal end, can then carry out the movement phase of the drive module 3 in order to insert the catheter into the arterial introducer.

As the double-articulated robotic arm 5 is fixed directly on the table 7, the use of an axis of rotation located at the base of the arm 5, and its association with the segments 50 and their joints 51, allows the distal end of the arm 5 carrying the drive module 3 to follow a rectilinear path when its proximal end located at the base of the arm 5 is actuated in rotation. Such an arm 5 allows a larger range of movement, and therefore it can manage both the adjustment of the end point of the catheter 1 and the path of the catheter 1.

FIG. 5C schematically represents a side view of a third exemplary arm supporting a catheter drive module in a catheter insertion robot according to one embodiment of the invention.

The catheter drive system of a catheter insertion robot comprises an articulated arm 5 comprising three segments 50 pivotally connected by joints 51, the arm 5 being robotic and able to trace a linear path through space at its distal end which carries a catheter drive module 3 integrally secured to said distal end. One position of the arm 5 is indicated with solid lines and another position of the arm 5 with dashed lines, to illustrate the movement of the arm 5 and of the drive module 3 from one position to another.

In order, from patient to table 7, we find the drive module 3 carried by a first segment 50 at the distal end of the arm 5, a second segment 50, then a third segment 50 connected at its proximal end to a support post 52 which is slidable along an adjustment rail 10 arranged on the table 7. The support post 52 comprises a vertical portion with a base that slides in the adjustment rail 10, and a horizontal portion with one side attached at the top of the vertical portion and the other side to the proximal end of the arm 5.

Once the support post 52 is immobilized along the adjustment rail 10 relative to the size and position of the patient and relative to the arrangement of the arterial introducer in the patient, the various segments 50 pivot to cause the drive module 3 to trace a linear path in order to insert the catheter into the arterial introducer by means of linear movement of the drive module 3.

The adjustment phase is carried out by moving the support post 52 along the adjustment rail 10, while the movement phase of the drive module 3 is carried out by deployment of the arm 5 as its segments 50 pivot about the joints 51.

As the robotic arm 5 is double-articulated but attached to the end of a support post 52, the mobility of this support post 52 ensures the adjustment of the end point of the catheter 1.

FIG. 5D schematically represents a side view of a fourth exemplary arm supporting a catheter drive module in a catheter insertion robot according to one embodiment of the invention.

The catheter drive system of a catheter insertion robot comprises an articulated arm 5 comprising four segments 50 pivotally connected together by joints 51, which is robotized and which is able to trace a linear path through space at its distal end which carries a catheter drive module 3 integrally secured to said distal end.

In order, from patient to table 7, we find the drive module 3 carried by a first segment 50 at the distal end of the arm 5, a second segment 50, a third segment 50, and a fourth segment 50 anchored to the floor 102 by an anchor 53, this fourth segment 50 being vertical.

The various segments 50 pivot to cause the drive module 3 to trace a linear path in order to insert the catheter into the arterial introducer by means of linear movement of the drive module 3.

The adjustment phase and the movement phase of the drive module 3 are carried out simultaneously by deployment of the arm 5, its segments 50 pivoting on the joints 51. Alternatively, a portion of the arm 5, nearer its proximal end, can first carry out the adjustment phase, while another portion of the arm 5, nearer its distal end, can then carry out the movement phase of the drive module 3 in order to insert the catheter into the arterial introducer.

Because the fully robotic arm 5 is placed on the floor 102, this drive system eliminates the issue of attachment to the table 7, but at the expense of needing an additional system for monitoring the movements of the table 7 in order to prevent the table 7 from being moved without the robotic arm 5.

FIG. 5E schematically represents a side view of a fifth exemplary arm supporting a catheter drive module in a catheter insertion robot according to one embodiment of the invention.

The catheter drive system of a catheter insertion robot comprises an articulated arm 5 comprising three segments 50 pivotally connected by joints 51, this robotic arm 5 being able to trace a linear path through space at its distal end which carries a catheter drive module 3 integrally secured to the distal end. One position of the arm 5 is indicated in solid lines and another position of the arm 5 in dashed lines, to illustrate movement of the arm 5 and drive module 3 from one position to another.

In order, from patient to table 7, we find the drive module 3 carried by a first segment 50 at the distal end of the arm 5, a second segment 50, a third segment 50 connected at its proximal end to an adjustment arm 54 which itself comprises a plurality of segments pivotally connected together.

Once the adjustment arm 54 is immobilized in a required position relative to the size and position of the patient as well as relative to the arrangement of the arterial introducer in the patient, the various segments 50 pivot to cause the drive module 3 to trace a linear path in order to insert the catheter into the arterial introducer by means of linear movement of the drive module 3.

The adjustment phase is carried out by moving the adjustment arm 54 as a whole, while the movement phase of the drive module 3 is carried out by deployment of the arm 5, its segments 50 pivoting on the joints 51. As the robotic arm 5 is double-articulated and attached to an adjustment arm 54, it is this adjustment arm 54 which makes it possible to adjust the end point of the catheter 1 relative to the arterial introducer. The seal between the consumable and non-consumable portions is ensured by a sterile sleeve fully covering the robotic arm 5.

FIGS. 5F and 5G schematically represent respective perspective views of a sixth exemplary arm supporting a catheter drive module in a catheter insertion robot according to one embodiment of the invention.

The catheter drive system of a catheter insertion robot comprises a robotic articulated arm 5 comprising three segments 50 pivotally connected by joints 51 which are connections pivoting about the vertical axis Z, able to trace a linear horizontal path in the XY plane at its distal end which carries a catheter drive module 3 integrally secured to the distal end.

In order, from patient to table 7, we find the drive module 3 carried by a first segment 50 at the distal end of the arm 5, a second segment 50, a third segment 50 connected at its proximal end 11 to a height-adjustable post, this post 11 being slidably mounted on an adjustment rail not shown in these figures but similar to the adjustment rail 10 shown in FIG. 5A.

Once the post 11 is adjusted in height and immobilized along the adjustment rail 10 relative to the size and position of the patient and relative to the arrangement of the arterial introducer in the patient, the various segments 50 pivot to cause the drive module 3 to trace a linear and horizontal path in order to introduce the catheter into the arterial introducer by means of linear movement of the drive module 3.

The adjustment phase is implemented by the height adjustment and movement of the post 11 carrying the arm 5 along the adjustment rail 10, while the movement phase of the drive module 3 is carried out by deployment of the arm 5, its segments 50 pivoting horizontally on the joints 51.

As the drive module is carried by a motorized arm 5 composed of multiple segments 50 pivoting horizontally about the joints 51, the translational movement of the drive module 3 is carried out by deploying the robotic arm 5 in only one horizontal plane. This reduces the masses to be moved for each portion of the robotic arm 5. The forces applied to the robotic arm 5 will therefore be reduced, and it will be easier to detect strain in the motorization of this robotic arm 5. The drive module 3 will be installed at the distal end of the robotic arm 5, therefore at the end opposite to its proximal end connected to the post 11. This post 11 could also be installed on an adjustment rail 10 of the table 7. This post 11 is then placed at a distance that enables maximum advancement and maximum withdrawal of the robotic arm 5 during the operation. In this case, the post 11 of course remains height-adjustable in order to accommodate different patient morphologies.

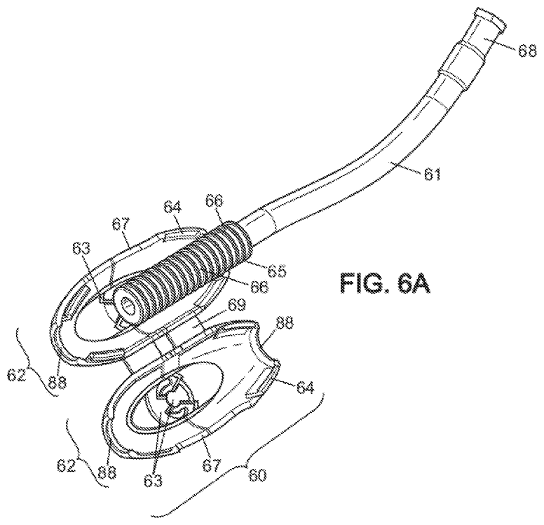

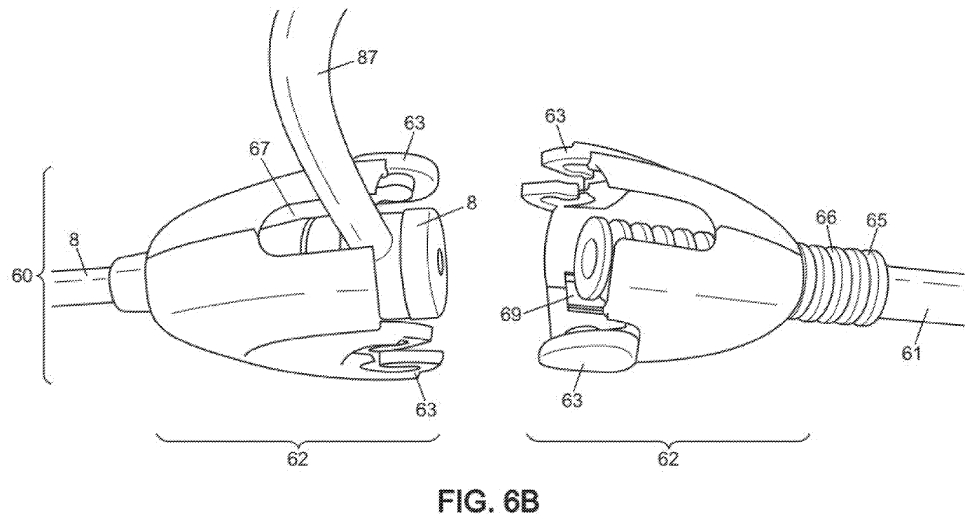

FIGS. 6A and 6B schematically represent perspective views, respectively in an assembled position and in a detached position, of an exemplary arterial introducer connector in a catheter insertion robot according to one embodiment of the invention.

The connector 60 comprises multiple parts 62. This connector 60 may be separated into multiple parts 62 in two ways, either on both sides of a longitudinal plane as shown in FIG. 6 A, or on both sides of a transverse plane as shown in FIG. 6B.

On one longitudinal side of the connector 60, the guide tube 61 terminated by a “luer lock” female fitting 68 enters via a longitudinal opening 88 towards the drive module. The “luer lock” fitting is defined in ISO 594, EN 1707:1996, and EN 20954-1: 1993. On the other longitudinal side of the connector 60, via a longitudinal opening 88, enters the arterial introducer 8 which is also connected to a lateral tube 87 which enters through a lateral opening.

A sleeve 65 comprises a plurality of ribs for adjusting the length of the sleeve 65 projecting into the connector 60, to ensure that the sleeve 65 preferably comes substantially in contact with the arterial introducer 8, and does so for different sizes and morphologies of the arterial introducer. This contact ensures continuity in the path of the catheter 1 from the guide tube 61 through the sleeve 65 and then into the arterial introducer 8, and does so without the risk of a loop forming in the connector 60, between the exit from the sleeve 65 and the entrance into the arterial introducer 8.

In FIG. 6A, the connector 60 is separated longitudinally into two parts 62 connected by hinges 69. By folding the two parts 62 one onto the other, the connector 60 will close at the side clips 64.

In FIG. 6B, the connector 60 is separated transversely into two parts 62. By bringing the two parts 62 against one another, the connector 60 will close at the central clips 63. The central clips 63 must be able to detach easily when forced or when there is sudden stress caused by abrupt movement of the arm or other limb of a patient, and in particular to detach more easily than the side clips 64. The male part of the clip 63 is a disk that fits into the female part which is V-shaped widening in a circle towards the apex of the V. The male part of the clip 63 also comprises a lateral flange to guide its engagement with the female part of the clip 63.

In summary, the connector 60 is placed at the exit of the drive module and enables the connection between the drive system and the arterial introducer 8. This connector 60 allows rapid assembly of this fitting onto the arterial introducer 8 already in place, serving as a guide conveying the guide 2 (not shown in FIGS. 6A and 6B) and or the catheter 1 while preventing them from looping in case of resistance to advancement. This connector 60 is above all a safety feature for when the patient moves suddenly, because it allows separation of the drive system and patient without pulling on the arterial introducer 8.

Rapid assembly of the fitting is then carried out as follows. The sleeve 65 is brought into near-contact with the already positioned arterial introducer 8 while performing the operation of moving the drive system closer and manually positioning it. Next, the previously assembled two parts 62 of the connector 60 (as shown in FIG. 6A) are closed onto the arterial introducer 8 and sleeve 65 to ensure they are held in place relative to one another.

In case of patient restlessness, for example if the patient suddenly pulls back, in order to avoid patient injury, the central clips 63 of the previously joined two parts 62 of the connector 60 will open (as shown in FIG. 6B) to allow these two parts 62 of the connector 60 to detach, thus freeing the patient from the drive system by the application of very little force on the arterial introducer 8.

Thus, the combination of the central clips 63 and side clips 64, with their respective tensile strengths, will allow disconnection in case of accidental excessive stresses in order to ensure patient safety, and also to allow disconnection which is not accidental for the purpose of inserting and removing the drive system, while the presence of the sleeve 65 will allow adjusting to different morphologies of the arterial introducer 8 and guarantee a good connection, meaning a connection leaving very little space between the arterial introducer 8 and the sleeve 65.

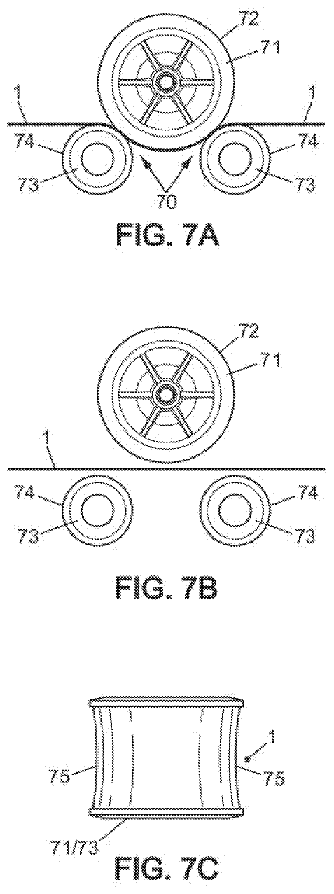

FIGS. 7A and 7B schematically represent top views of an exemplary set of guide rollers of a catheter drive module in a catheter insertion robot according to one embodiment of the invention.

The catheter drive module of a catheter insertion robot comprises three rotatable rollers 71 and 73 which are movable relative to one another so as to come closer together to form a deflector 70 between them during the passage of the catheter 1. The small rollers 73 have a periphery 74 and the large roller 71 has a periphery 72.

In FIG. 7A, the two small pressure rollers 73 and the large drive 71 are closer to each other and to the catheter 1. The periphery 74 of the small pressure rollers 73 becomes tangential to the periphery 72 of the large drive roller 71, gripping the catheter 1. The catheter 1 must first pass between the periphery 74 of a small pressure roller 73 and the periphery 72 of the large drive roller 71, then follows the periphery 72 of the large drive roller 71, and then passes between the periphery 74 of the other small pressure roller 73 and the periphery 72 of the large drive roller 71, before continuing in the same direction as its direction when it arrived.

This route forms the deflector 70 within which the catheter 1 arrives in an arrival direction, changes direction, and then changes direction again before exiting the deflector 70 in a departure direction parallel to the arrival direction, and even coincident with its arrival direction. The large drive roller 71 drives the catheter 1 in order to advance it. The small pressure rollers 73 retain the catheter 1 against the large drive roller 71.

In FIG. 7B, the two small pressure rollers 73 and the large drive roller 71 have moved away from each other and from the catheter 1. The periphery 74 of the small pressure rollers 73 and the periphery 72 of the large drive roller 71 no longer touch the catheter 1 which has resumed its completely linear direction. The deflector 70 has disappeared.

With the large drive roller 71 which is placed facing the two pressure rollers 72, the curvature generated on the guide 2 or on the catheter 1 by the positions of these three rollers provides good translation and good rotation, due to a crank-like effect. This entire system can be opened in order to introduce the element to be driven (guide 2 or catheter 1). This roller system has fewer components in general than a belt system known from the prior art.

FIG. 7C schematically represents a side view of an exemplary drive roller in a set of guide rollers of a catheter drive module in a catheter insertion robot according to one embodiment of the invention.

The two small pressure rollers 73 and the large drive roller 71 have a centered concave portion 75 for guiding the catheter 1. Thus, the catheter 1, as it passes through the deflector 70, remains guided in the center of the concave portions 75 of the rollers 71 and 73, and no longer runs the risk of leaving the guidance of the rollers 71 and 73.

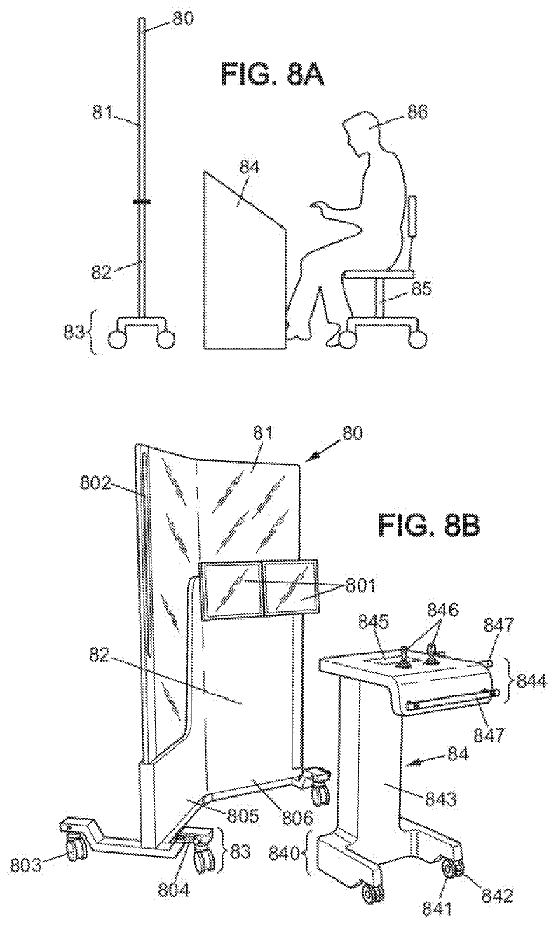

FIG. 8A schematically represents a side view of an exemplary remote control station for a catheter drive module in a catheter insertion robot, incorporating a radioprotective shield, according to one embodiment of the invention.

This cockpit which consists of two separate elements, namely the control station (also called the control desk 84) and the protective shield 80, allows the user who is the physician 86 to control the remote-controlled robot from an area protected from X-rays during an intervention for example in an angiography room.

The physician 86, sitting on a chair 85, remotely controls the advancement of the catheter in the patient, from the control desk 84 which is at a distance from the patient lying on a table. To minimize radiation exposure by the physician 86, a protective shield 80 isolates the physician 86 from the table and patient. It is critical to reduce the radiation received by the physician 86, because he or she is exposed to it all day long, unlike the patient who is only exposed for the duration of the examination and for whom it is important to reduce the radiation but less critical.

The shield 80, independent of the control desk 84 as is the table where the patient is lying, protects the physician 86 from radiation because it is opaque to this radiation, usually X-rays. The shield 80 comprises an upper portion 81 and a lower portion 82, both radiopaque. The upper portion 81 is see-through, meaning it is transparent to visible light, enabling the physician 86 to monitor the patient remotely. The lower portion 82 is opaque to visible light as well, therefore being simpler to implement. The height of the lower portion 82 is approximately 50 cm. The shield 80 is movable and mounted on wheels 83, so it can easily be moved around the room.

The fact that the protective shield 80 is independent of the control desk 84 optimizes its integration into a room, facilitating access and placement of the shield 80 against a wall after use or during use with no X rays.

The fact that the protective shield 80 is advantageously a single part, ensures protection over the entire surface of the protective shield 80 because there is then no cutout, hinge, or hole.

FIG. 8B schematically represents a perspective view of another exemplary remote control station for a catheter drive module in a catheter insertion robot, incorporating a radioprotective shield according to one embodiment of the invention.

The protective shield 80 comprises all or part of the following: a see-through area 81 transparent to visible light, an area 82 opaque to visible light, wheels 803 with brakes 804, multiple handles 802 arranged to allow one person to roll the protective shield 80 over the floor, preferably means (not shown in FIG. 8B because located behind the display screens) for hanging display screens 801, for example such as screens to duplicate angiography images provided with means for adjusting the height and/or width, preferably means (not shown in FIG. 8B) for hanging cables.

The protective shield 80 is one piece but comprises two plates 805 and 806 which are not parallel to one another, even if they form one piece.

The control station 84 comprises all or part of the following: wheels 841 with brakes 842, at least one controller 846, preferably a joystick 846, at least one monitoring screen 845, preferably liquid crystal, preferably touchscreen, at least one other human-machine interface, comprising buttons and/or indicator lights which are preferably light-emitting diodes, preferably accessory hooks 847, said accessories being for example a remote control for contrast agent injection, a control for an examination table and/or a C-arm for angiography, a balloon pump.

On the work desk 844 of the control station 84, the monitoring screen 845 reports information on the operation of the insertion robot in response to commands given to this insertion robot using controllers 846, for example joysticks, one for translation of the insertion robot and the other for rotation of the insertion robot. This work desk 844 rests on the body 843 of the control station 84, and also comprises hooks 847 for accessories. Included among the accessories which can be hung thereon are controls for a C-arm or table for angiography, a remote control for contrast medium injection, a balloon pump located at the end of the catheter and used to inflate the balloon.

Of course, the present invention is not limited to the examples and to the embodiment described and represented, but is capable of many variants accessible to the skilled person.

### Remainder of Description: Other Objects of the Invention

Object 1) Elongate flexible medical instrument drive system of a robot for insertion of an elongate flexible medical instrument, comprising:
an arm (5),
a non-motorized linear rail (4) carried by the arm (5), a motorized drive module (3) for an elongate flexible medical instrument (1), which slides along the linear rail (4).

Object 2) Elongate flexible medical instrument drive system according to object 1, wherein:
the elongate flexible medical instrument motorized drive module (3) comprises two parts (14, 15) which can be separated from one another:
   a reusable motor (14) without contact with the linear rail (4),
   a disposable carriage (15) sliding on the linear rail (4), this carriage (15) preferably being for one-time use.

Object 3) Elongate flexible medical instrument drive system according to object 2, wherein:
the sliding of the disposable carriage (15) on the linear rail (4) creates the translational movement of the elongate flexible medical instrument (1).

Object 4) Elongate flexible medical instrument drive system according to any one of objects 2 to 3, wherein:
the disposable carriage (15) comprises a contact surface with the linear rail (4), said contact surface being E-shaped so that the disposable carriage (15) rests on three of the four sides of the linear rail (4).

Object 5) Elongate flexible medical instrument drive system according to any one of the above objects, wherein:
the linear rail (4) is disposable, and preferably the linear rail (4) is for one-time use.

Object 6) Elongate flexible medical instrument drive system according to any one of the above objects, wherein it further comprises:
a consumable sterile barrier (6) passing between the reusable motor (14) and the disposable carriage (15) which are integrally secured together.

Object 7) Elongate flexible medical instrument drive system according to object 6, wherein:
this sterile barrier (6) comprises a plate which is perforated so as to allow the passage of couplings between the disposable carriage (15) and reusable motor (14) and which is surrounded by a film attached to the edges of the plate.

Object 8) Elongate flexible medical instrument drive system according to object 6, wherein:
this sterile barrier (6) includes the disposable carriage (15) which is surrounded by a film attached to the edges of the disposable carriage (15).

Object 9) Elongate flexible medical instrument drive system according to any one of the foregoing objects, wherein it further comprises:
another consumable sterile barrier (6) encompassing the entire arm (5), but neither the linear rail (4) nor the elongate flexible medical instrument drive module (3)

Object 10) Elongate flexible medical instrument drive system according to any one of the foregoing objects, wherein:
the path length of the motorized module (3) along the linear rail (4) is between 60 cm and 120 cm.

Object 11) Elongate flexible medical instrument drive system according to any one of the foregoing objects, wherein:
the linear rail (4) comprises at least one groove (18) guiding the elongate flexible medical instrument (1), preferably the catheter (1) and also the guide (2).

Object 12) Elongate flexible medical instrument drive system according to object 11, wherein:
the groove (18) is closed by a cover (19) which opens when the motorized module (3) passes by and which closes after the passage of the motorized module (3).

Object 13) Elongate flexible medical instrument drive system according to any one of the foregoing objects, wherein the arm (5) comprises:
a post (11) that is movable in vertical translation and in horizontal translation,
two bars forming a V, the apex of the V preferably comprising a ball joint (12) coupling it to the top of the movable post (11), the free ends of the V preferably being fixedly connected to the linear rail (4).

Object 14) Elongate flexible medical instrument drive system according to any one of the foregoing objects, wherein:
the drive system comprises members for locking the assembly formed by the arm (5), linear rail (4) and motorized module (3), such that this assembly is movable as a unit relative to the operating table (7).

Object 15) Elongate flexible medical instrument drive system according to any one of the foregoing objects, wherein:
the motorized drive module (3) carries a catheter (1) drive module and a guide (2) drive module for driving in translation and in rotation.

Object 16) Elongate flexible medical instrument drive system according to any one of the foregoing objects, wherein:
the motorized drive module (3) for the elongate flexible medical instrument (1) is controlled by a wireless link and/or has one or more electric batteries as the primary and preferably exclusive energy source.

Object 17) Elongate flexible medical instrument drive system according to any one of the foregoing objects, wherein:
the elongate flexible medical instrument is a catheter (1) and/or a guide (2).

Object 18) Elongate flexible medical instrument drive system of a robot for insertion of an elongate flexible medical instrument, comprising:
an arm (5),
a motorized linear rail (4) carried by the arm (5),
an elongate flexible medical instrument drive module (3) which slides along the linear rail (4) under the effect of the motorization of only the linear rail (4).

Object 19) Elongate flexible medical instrument drive system according to object 18, wherein:
the elongate flexible medical instrument drive module (3) comprises two parts (14, 15) which can be detached from one another:
   a reusable carriage sliding on the linear rail (4),
   a disposable support without contact with the linear rail (4), said support preferably being for one-time use, driving the elongate flexible medical instrument, preferably the catheter (1) and preferably also the guide (2).

Object 20) Elongate flexible medical instrument drive system according to any one of objects 18-19, wherein it further comprises:
a consumable sterile skirt (6) passing between the reusable carriage and the disposable support which are integrally secured together.

Object 21) Elongate flexible medical instrument drive system according to object 20, wherein it further comprises:
the consumable sterile barrier, passing between the reusable carriage and the disposable support which are integrally secured together, which also encompasses the entire arm (5).

Object 22) Elongate flexible medical instrument drive system according to any one of objects 18 to 21, wherein:
the motorized drive module (3) comprises a catheter (1) drive module and a guide (2) drive module for driving in translation and in rotation.

Object 23) Elongate flexible medical instrument drive system according to any one of objects 18 to 22, wherein:
the elongate flexible medical instrument is a catheter (1) and/or a guide (2).

Object 24) Method for creating a sterile barrier (6) between the consumable and non-consumable parts of an elongate flexible medical instrument drive system of a robot for insertion of an elongate flexible medical instrument, comprising a step of installing a consumable sterile skirt (6) separating a linear rail (4) from at least a portion of an elongate flexible medical instrument drive module (3) in said elongate flexible medical instrument drive system.

Object 25) Consumable sterile skirt adapted to separate a linear rail (4) from at least a portion of an elongate flexible medical instrument drive module (3) in an elongate flexible medical instrument drive system of a robot for insertion of an elongate flexible medical instrument, thereby providing a sterile barrier (6) between the consumable and non-consumable parts of said elongate flexible medical instrument drive system.

Object 26) Elongate flexible medical instrument drive system of a robot for insertion of an elongate flexible medical instrument, comprising a sterile barrier (6) between its consumable and non-consumable parts, comprising:
a linear rail (4),
an elongate flexible medical instrument drive module (3),
a consumable sterile skirt (6) separating the linear rail (4) from at least a portion of the elongate flexible medical instrument drive module (3).

Object 27) Elongate flexible medical instrument drive system according to object 26, wherein:
the skirt (6) is longitudinally wrinkled, on each side of the elongate flexible medical instrument drive module (3), so as to maintain the sterility barrier (6) for the entire translational path of the elongate flexible medical instrument drive module (3) along the linear rail (4).

Object 28) Elongate flexible medical instrument drive system according to object 27, wherein:
the wrinkled skirt (6) comprises lateral elastic members (31) to hold the wrinkled skirt (6) around the linear rail (4).

Object 29) Consumable sterile skirt according to object 25, wherein:
the skirt (6) is longitudinally wrinkled, on each side of a central portion corresponding to an attachment at the elongate flexible medical instrument drive module (3), so as to maintain the sterility barrier (6) for the entire translational path of the elongate flexible medical instrument drive module (3) along the linear rail (4), the wrinkled skirt (6) advantageously comprising lateral elastic members (31) to hold this wrinkled skirt (6) around the linear rail (4).

Object 30) Elongate flexible medical instrument drive system according to object 26, wherein:
the skirt (6) is longitudinally slit while having one side of the slit overlap the other side of the slit so as to maintain a sterility channel around the linear rail (4).

Object 31) Elongate flexible medical instrument drive system according to object 30, wherein:
the slot is opened in response to a spreading front shape (100) of the elongate flexible medical instrument drive module (3) and is closed in response to a closing rear shape (101) of the elongate flexible medical instrument drive module (3).

Object 32) Elongate flexible medical instrument drive system according to object 26, wherein:
the skirt (6) is longitudinally slit with the sides of the slit lying edge-to-edge, to maintain a sterility channel around the linear rail (4).

Object 33) Elongate flexible medical instrument drive system according to object 32, wherein:
the slit is opened in response to a spreading front shape (100) of the elongate flexible medical instrument drive module (3) and is closed in response to a closing rear shape (101) of the elongate flexible medical instrument drive module (3).

Object 34) Elongate flexible medical instrument drive system according to any one of objects 32 to 33, wherein it further comprises:
a pouch surrounding the elongate flexible medical instrument drive module (3).

Object 35) Elongate flexible medical instrument drive system according to object 26, wherein it comprises:
a first winder/unwinder (34) integrally secured to a first end of the elongate flexible medical instrument drive module (3),
a first consumable sterile skirt (6) immobilized on one side relative to a first end of the linear rail (4), and located on another side in the first winder/unwinder (34) so as to be able to respectively wind or unwind depending on the direction of movement of the elongate flexible medical instrument drive module (3) along the linear rail (4), the first consumable sterile skirt (6) being integrally secured on one side to the first end of the linear rail (4),
a second winder/unwinder (34) integrally secured to a second end of the elongate flexible medical instrument drive module (3),
a second consumable sterile skirt (6) immobilized on one side relative to a second end of the linear rail (4) and located on another other side in the second winder/unwinder (34) so as to be able to respectively wind or unwind while the first consumable sterile skirt (6) unwinds or winds, the second consumable sterile skirt (6) being integrally secured on one side to the second end of the linear rail (4).

Object 36) Elongate flexible medical instrument drive system according to object 26, wherein:
the consumable sterile skirt (6) has a length which is at least twice the path length of the elongate flexible medical instrument drive module (3) along the linear rail (4).

Object 37) Elongate flexible medical instrument drive system according to object 36, wherein:
the consumable sterile skirt (6) is smooth over its entire surface.

Object 38) Elongate flexible medical instrument drive system according to object 36 or 37, wherein it comprises:
an arterial introducer (8),
a cowl (36) located on the side closest to the arterial introducer (8), arranged so as to bring the consumable sterile skirt (6) from the opposite side relative to that of the elongate flexible medical instrument drive module (3) with respect to the sliding plane of the linear rail (4).

Object 39) Elongate flexible medical instrument drive system according to object 26, wherein:
the consumable sterile skirt (37) is fixed to the linear rail,
and wherein the elongate flexible medical instrument drive system comprises another consumable sterile skirt (6) that covers both the linear rail (4) and the elongate flexible medical instrument drive module (3).

Object 40) Elongate flexible medical instrument drive system according to object 26, wherein:
the consumable sterile skirt (6) is arranged around the linear rail (4) so as to be rotated about the linear rail (4), about an axis perpendicular to the longitudinal axis of the linear rail (4), when the elongate flexible medical instrument drive module (3) moves along the linear rail (4).

Object 41) Elongate flexible medical instrument drive system according to object 40, wherein:
the consumable sterile skirt (6) is weighted so as to remain around the linear rail (4).

Object 42) Elongate flexible medical instrument drive system according to object 40 or 41, wherein it comprises:
flanges to guide the rotation of the consumable sterile skirt (6) about the linear rail (4).

Object 43) Elongate flexible medical instrument drive system according to any one of objects 24 to 42, wherein:
the elongate flexible medical instrument is a catheter (1) and/or a guide (2).

Object 44) Elongate flexible medical instrument drive system, wherein it comprises:
an elongate flexible medical instrument (1),
an elongate flexible medical instrument drive module (3) whose movement causes movement of the elongate flexible medical instrument (1), preferably by pushing the elongate flexible medical instrument (1),
an arterial introducer (8),
a track (40) to guide the elongate flexible medical instrument between the elongate flexible medical instrument drive module (3) and the arterial introducer (8).

Object 45) Elongate flexible medical instrument drive system according to object 44, wherein:
the guide track (40) is structured to open and then preferably to close when the elongate flexible medical instrument drive module (3) passes by.

Object 46) Elongate flexible medical instrument drive system according to object 45, wherein:
the guide track (40) is a slit tube (43).

Object 47) Elongate flexible medical instrument drive system according to object 45, wherein:
the guide track (40) is closed by a slide fastener (45) or by a zipper (45).

Object 48) Elongate flexible medical instrument drive system according to object 44 or 45, wherein:
the guide track (40) is a section (400) which is flexible when open and which is rigid when refolded and closed.

Object 49) Elongate flexible medical instrument drive system according to object 48, wherein the section comprises:
a bottom (401),
two longitudinal side members (402) respectively connected to the bottom (401) and pivotally connected to said bottom (401),
two closure members (403, 404) respectively located on the two longitudinal side members (402), able to engage each other to close the section (400), the closed cavity of the section (400) then being bounded by the bottom (401), the two longitudinal side members (402), and the two closure members (403, 404).

Object 50) Elongate flexible medical instrument drive system according to object 49, wherein:
the cross-sectional dimensions of the side members (402) and bottom (401) of the section (400) are determined so that:
   the section (400) is self-supporting when the closure members (403, 404) are closed, and
   the section (400) is not self-supporting when the closure members (403, 404) are open.

Object 51) Elongate flexible medical instrument drive system according to object 49 or 50, wherein:
the closure members (403, 404) are coupled by clipping one to the other.

Object 52) Elongate flexible medical instrument drive system according to any one of objects 49 to 51, wherein:
the pivoting connections are thinned areas (405) in the material.

Object 53) Elongate flexible medical instrument drive system according to object 52, wherein:
the thinned areas (405) in the material are notches which each have parallel beveled edges (406).

Object 54) Elongate flexible medical instrument drive system according to object 53, wherein:
each notch has a wide bottom (407) relative to the width between the beveled edges (406).

Object 55) Elongate flexible medical instrument drive system according to object 44, wherein:
the guide track (40) is in the form of a drag chain (41) having a longitudinal opening which is of smaller width than the diameter of the elongate flexible medical instrument (1) and which is flexible but asymmetrical to allow the elongate flexible medical instrument (1) to more easily enter than exit.

Object 56) Elongate flexible medical instrument drive system according to object 44, wherein:
the guide track (40) has the shape of a spiral (46) winding around the elongate flexible medical instrument (1), said spiral (46) being rotatable about the elongate flexible medical instrument (1).

Object 57) Elongate flexible medical instrument drive system according to object 44, wherein:
the guide track (40) comprises two parts (49) which are fixed at one end to the elongate flexible medical instrument drive module (3), are respectively fixed at the other end to the interior of two winders (48), and form a single band outside these two winders (48) within which they respectively wind as the elongate flexible medical instrument drive module (3) slides towards the arterial introducer (8).

Object 58) Elongate flexible medical instrument drive system according to object 44, wherein:
the guide track (40) comprises two crenellated rectangular parts (410) which are flexible when separated from one another and which form a rigid channel of rectangular cross-section when fitted one into the other.

Object 59) Elongate flexible medical instrument drive system according to object 44, wherein:
the guide track (40) is in the form of a bellows (412).

Object 60) Elongate flexible medical instrument drive system according to object 44, wherein the guide track comprises:
an open and rigid guide channel in the recess of which the elongate flexible medical instrument (1) is to be placed,
a flexible cover (414) which is fixed at one end to the elongate flexible medical instrument drive module (3) and is fixed at the other end to the interior of a winder (48) into which it is wound as the elongate flexible medical instrument drive module (3) slides towards the arterial introducer (8).

Object 61) Elongate flexible medical instrument drive system according to any one of objects 44 to 60, wherein:
the elongate flexible medical instrument is a catheter (1) and/or a guide (2).

Object 62) Elongate flexible medical instrument drive system of a robot for insertion of an elongate flexible medical instrument, comprising:
an articulated arm (5) comprising at least three segments (50) pivotally connected together and robotized, able to trace a linear path through space at its distal end,
an elongate flexible medical instrument drive module (3) integrally secured to this distal end.

Object 63) Elongate flexible medical instrument drive system according to object 62, wherein:
the spatial orientation of the elongate flexible medical instrument drive module (3) is kept constant during its movement along said linear path.

Object 64) Elongate flexible medical instrument drive system according to object 62, wherein:
this linear path through space remains within a horizontal plane, in other words in a plane parallel to the plane of the examination table (7).

Object 65) Elongate flexible medical instrument drive system according to any one of objects 62 to 64, wherein:
the arm (5) comprises at least four segments (50) pivotally connected together, preferably only four segments (50) pivotally connected together.

Object 66) Elongate flexible medical instrument drive system according to any one of objects 62 to 65, wherein it further comprises:
an adjustment rail (10) carrying the proximal end of said arm (5),
a device for locking this proximal end of the arm (5) on the adjustment rail (10) during linear movement of this distal end.

Object 67) Elongate flexible medical instrument drive system according to object 66, wherein:
the adjustment rail (10) rests on an examination table (7), preferably being fixed to said examination table (7).

Object 68) Elongate flexible medical instrument drive system according to any one of objects 62 to 65, wherein:
the proximal end of said arm (5) rests on an examination table (7), pivotally connected by a rotary connection to said examination table (7), advantageously pivotally connected only by said rotary connection to said examination table (7).

Object 69) Elongate flexible medical instrument drive system according to any one of objects 62 to 65, wherein it further comprises:
an adjustment rail (10) carrying a non-articulated support post (52) to which the proximal end of said arm (5) is integrally secured,
a device for locking said support post (52) on the adjustment rail (10) during linear movement of said distal end.

Object 70) Elongate flexible medical instrument drive system according to object 69, wherein:
the adjustment rail (10) rests on an examination table (7), preferably being fixed to said examination table (7).

Object 71) Elongate flexible medical instrument drive system according to any one of objects 62 to 65, wherein it further comprises:
a non-articulated support post resting on the floor (102), to which the proximal end of said arm (5) is integrally secured,
a device slaving it to the movement of an operating table (7) during linear movement of said distal end.

Object 72) Elongate flexible medical instrument drive system according to object 71, wherein:
the support post rests (53) on the floor (102).

Object 73) Elongate flexible medical instrument drive system according to object 71 or 72, wherein:
the support post is higher than the examination table (7) associated with the elongate flexible medical instrument drive system.

Object 74) Elongate flexible medical instrument drive system according to any one of objects 62 to 65, wherein it further comprises:
an articulated adjustment arm (5) carrying the proximal end of said robotic arm (5),
a device for locking said articulated adjustment arm (54) during linear movement of said distal end.

Object 75) Elongate flexible medical instrument drive system according to object 74, wherein:
the articulated adjustment arm (54) rests on an examination table (7), preferably being fixed to said examination table (7).

Object 76) Elongate flexible medical instrument drive system according to object 74 or 75, wherein:
the articulated adjustment arm (54) comprises at least three segments (50) pivotally connected together.

Object 77) Elongate flexible medical instrument drive system according to any one of objects 62 to 65, wherein it further comprises:
a post (11) to which the proximal end of said articulated robotic arm (5) is integrally secured,
and wherein all segments (50) of the articulated robotic arm (5) only deploy within a horizontal plane (X, Y).

Object 78) Elongate flexible medical instrument drive system according to object 77, wherein:
the post (11) is not articulated.

Object 79) Elongate flexible medical instrument drive system according to object 77 or 78, wherein it further comprises:
an adjustment rail (10) on which the post (11) rests,
a device for locking said post (11) on the adjustment rail (10) during linear movement of said distal end.

Object 80) Elongate flexible medical instrument drive system according to object 79, wherein:
the adjustment rail (10) rests on an examination table (7).

Object 81) Elongate flexible medical instrument drive system according to any one of objects 62-80, wherein:
the elongate flexible medical instrument is a catheter (1) and/or a guide (2).

Object 82) Connector between arterial introducer (8) and catheter guide tube (61) of a robot for insertion of an elongate flexible medical instrument,
comprising two parts (62) interconnected by at least a first fastener (63), maintaining the arterial introducer (8) as an extension of the guide tube (61) to allow the passage of the elongate flexible medical instrument (1) from the guide tube (61) to the arterial introducer (8) by pushing the elongate flexible medical instrument (1),
the tensile strength, along the axis of the elongate flexible medical instrument (1) traversing the connector (60), of the first fastener (63) prior to releasing one of the two parts (62) of the connector (60) from the other, is less than that of the arterial introducer (8) inserted into the patient (9) before it emerges.

Object 83) Connector between arterial introducer and guide tube of the elongate flexible medical instrument of a robot for insertion of an elongate flexible medical instrument,
comprising four parts (62) interconnected by at least a first fastener (63), maintaining the arterial introducer (8) as an extension of the guide tube (61) to allow the passage of the elongate flexible medical instrument (1) from the guide tube (61) to the arterial introducer (8) by pushing the elongate flexible medical instrument (1), said first fastener (63) connecting two of the parts (62) located on one side of a transverse plane with the other two parts (62) located on the other side of this transverse plane,
comprising at least a second fastener (64) working with the first fastener (63) so that together they immobilize the four parts (62) of the connector (60), the arterial introducer (8), and the guide tube (61), said second fastener (64) connecting two of the parts (62) located on one side of a longitudinal plane with the other two parts (62) located on the other side of this longitudinal plane,
the tensile strength, along the axis of the elongate flexible medical instrument (1) traversing the connector (60), of the first fastener (63) prior to its release, is less than that of the second fastener (64) prior to its release.

Object 84) Connector according to object 83, wherein:
the second fastener (64) works together with a flexible hinge (69) which facilitates the opening and closing of the second fastener (64).

Object 85) Connector according to any one of objects 82 to 84, wherein:
the first fastener (63) comprises at least one central clip, and preferably a plurality of longitudinal clips,
the second fastener (64) comprises at least one side clip, and preferably a plurality of side clips.

Object 86) Connector according to any one of objects 82 to 85, wherein:
the second fastener (64) integrally secures the guide tube (61) to the connector (60) by means of a sleeve (65) surrounding the guide tube (61), this sleeve (65) being held in place in the connector (60) by the second fastener (64) at several positions along the axis of said sleeve (65).

Object 87) Connector according to object 86, wherein:
the sleeve (65) has ribs (66) along its axis.

Object 88) Connector according to object 87, wherein:
these ribs (66) are arranged periodically along the axis of the sleeve (65).

Object 89) Connector according to object 87 or 88, wherein:
the number of ribs (66) is between 5 and 15, preferably equal to 10, the dimension of the recess as well as the hump of each rib (66) being between 0.5 mm and 2 mm, preferably equal to 1 mm.

Object 90) Connector according to any one of objects 86 to 89, wherein:
said sleeve (65) is permanently fixed to the guide tube (61) which it surrounds.

Object 91) Connector according to any one of objects 82 to 90, wherein:
the connector (60) comprises a lateral opening (67) enabling the insertion, into the connector (60), of another tube (87) coming from the arterial introducer (8).

Object 92) Connection system comprising:
a connector (60) according to any one of objects 82 to 91,
an arterial introducer (8),
a guide tube (61) for the elongate flexible medical instrument (1) of a robot for insertion of an elongate flexible medical instrument.

Object 93) Connection system according to object 92, wherein it comprises:
a catheter (1) and a guide (2) which are coaxial.

Object 94) Connection system according to any one of objects 82 to 93, wherein:
the elongate flexible medical instrument is a catheter (1) and/or a guide (2).

Object 95) Elongate flexible medical instrument drive module of a robot for insertion of an elongate flexible medical instrument, comprising:
at least three rotatable rollers (71, 73), preferably only three rotatable rollers (71, 73), movable relative to one another so as to come closer together to form a deflector (70) between them during the passage of the elongate flexible medical instrument (1).

Object 96) Elongate flexible medical instrument drive module according to object 95, wherein:
at least one of the three rotatable rollers is a drive motor roller, and preferably only one of the three rollers is a drive motor roller.

Object 97) Elongate flexible medical instrument drive module according to object 95 or 96, wherein:
the axes of rotation of the rotatable rollers (71, 73) are parallel to each other and the rollers (71, 73) are circular in a plane perpendicular to their axes of rotation.

Object 98) Elongate flexible medical instrument drive module according to object 97, wherein:
the peripheries (74) of two rollers (73) are tangent to the periphery (72) of a third roller (71).

Object 99) Elongate flexible medical instrument drive module according to object 98, wherein:
the third roller (71) has a larger diameter than that of the other two rollers (73).

Object 100) Elongate flexible medical instrument drive module according to object 99, wherein:
the third roller (71) is a roller (71) for driving the elongate flexible medical instrument (1), while the two other rollers (73) are pressure rollers (73) which press the elongate flexible medical instrument (1) against the driving third roller (71).

Object 101) Elongate flexible medical instrument drive module according to any one of objects 98 to 100, wherein:
the other two rollers (73) have the same diameter as each other.

Object 102) Elongate flexible medical instrument drive module according to any one of objects 97 to 101, wherein:
in a plane perpendicular to the axes of rotation of the rollers (71, 73), the created angle whose apex is the center of the third roller (71) and which is formed by the two straight lines respectively connecting the centers of the two other rollers (73) to the center of the third roller (71), is between 60 degrees and 120 degrees, and is preferably about 90 degrees.

Object 103) Elongate flexible medical instrument drive module according to any one of objects 95 to 102, wherein:
at least the third roller (71), and preferably also the other two rollers (73), has (have) a concave portion (75) for centering and guiding the elongate flexible medical instrument (1).

Object 104) Elongate flexible medical instrument drive module according to any one of objects 95 to 103, wherein:
the concavity of said portion (75) is between one quarter and three quarters of the diameter of the elongate flexible medical instrument (1), preferably equal to half the diameter of the elongate flexible medical instrument (1).

Object 105) Elongate flexible medical instrument drive system comprising:
an elongate flexible medical instrument drive module (3) of a robot for insertion of an elongate flexible medical instrument according to any one of objects 95 to 104,
an elongate flexible medical instrument (1) passing through the deflector (70) formed by the rollers (71, 73).

Object 106) Elongate flexible medical instrument drive system according to any one of objects 95 to 105, wherein:
the elongate flexible medical instrument is a catheter (1) and/or a guide (2).

Object 107) Remote control cockpit for a robot for insertion of an elongate flexible medical instrument, comprising:
a control station (84) for said robot for insertion of an elongate flexible medical instrument, without an integrated radioprotective shield,
a radioprotective shield (80) that is independent of said control station (84).

Object 108) Cockpit according to object 107, wherein:
said protective shield (80) is movable on the floor, preferably said protective shield (80) rolls on the floor.

Object 109) Cockpit according to object 107 or 108, wherein:
said control station (84) is movable on the floor, preferably said control station (84) rolls on the floor.

Object 110) Cockpit according to any one of objects 107 to 109, wherein:
at least a portion of the surface of said protective shield (80) is transparent to visible light, preferably for its entire width and for more than the upper half of its height.

Object 111) Cockpit according to any one of objects 107 to 110, wherein:
said protective shield (80) is a single piece.

Object 112) Cockpit according to any one of objects 107 to 11, wherein:
said protective shield (80) comprises at least two planes which are not parallel to each other.

Object 113) Cockpit according to any one of objects 107 to 112, wherein said protective shield (80) comprises some or all of the following:
a see-through region (81) transparent to visible light,
a region (82) opaque to visible light,
wheels (803) with brakes (804)
multiple handles (802) arranged to enable one person to roll the protective shield (80) on the floor,
preferably, means for hanging display screens, for example screens to duplicate angiography images provided with means for height and/or width adjustment,
preferably, means for hanging cables.

Object 114) Cockpit according to any one of objects 107 to 113, wherein said control station (84) comprises some or all of the following:
wheels (841) with brakes (842)
at least one control member (846), preferably a joystick,
at least one monitoring screen (845), preferably liquid crystal, preferably touchscreen,
at least one other human-machine interface, comprising buttons and/or indicator lights which are preferably light-emitting diodes,
preferably hooks (847) for accessories, said accessories being for example a remote control for contrast agent injection, a control for an examination table and/or for a C-arm for angiography, a balloon pump.

Object 115) Elongate flexible medical instrument drive system according to any one of objects 107 to 114, wherein:
the elongate flexible medical instrument is a catheter (1) and/or a guide (2).

1-115. (canceled)

116. A connector between arterial introducer (8) and catheter guide tube (61) of a robot for insertion of an elongate flexible medical instrument,
comprising two parts (62) interconnected by at least a first fastener (63), maintaining the arterial introducer (8) as an extension of the guide tube (61) to allow the passage of the elongate flexible medical instrument (1) from the guide tube (61) to the arterial introducer (8) by pushing the elongate flexible medical instrument (1),
the tensile strength, along the axis of the elongate flexible medical instrument (1) traversing the connector (60), of the first fastener (63) prior to releasing one of the two parts (62) of the connector (60) from the other, is less than that of the arterial introducer (8) inserted into the patient (9) before it emerges.

117. A connector between arterial introducer and guide tube of the elongate flexible medical instrument of a robot for insertion of an elongate flexible medical instrument,
comprising four parts (62) interconnected by at least a first fastener (63), maintaining the arterial introducer (8) as an extension of the guide tube (61) to allow the passage of the elongate flexible medical instrument (1) from the guide tube (61) to the arterial introducer (8) by pushing the elongate flexible medical instrument (1), said first fastener (63) connecting two of the parts (62) located on one side of a transverse plane with the other two parts (62) located on the other side of this transverse plane,
comprising at least a second fastener (64) working with the first fastener (63) so that together they immobilize the four parts (62) of the connector (60), the arterial introducer (8), and the guide tube (61), said second fastener (64) connecting two of the parts (62) located on one side of a longitudinal plane with the other two parts (62) located on the other side of this longitudinal plane,
the tensile strength, along the axis of the elongate flexible medical instrument (1) traversing the connector (60), of the first fastener (63) prior to its release, is less than that of the second fastener (64) prior to its release.

118. The connector according to claim 117, wherein:
the second fastener (64) works together with a flexible hinge (69) which facilitates the opening and closing of the second fastener (64).

119. The connector according to claim 117, wherein:
the first fastener (63) comprises at least one central clip,
the second fastener (64) comprises at least one side clip.

120. The connector according to claim 117, wherein:
the second fastener (64) integrally secures the guide tube (61) to the connector (60) by means of a sleeve (65) surrounding the guide tube (61), this sleeve (65) being held in place in the connector (60) by the second fastener (64) at several positions along the axis of said sleeve (65).

121. The connector according to claim 120, wherein:
the sleeve (65) has ribs (66) along its axis.

122. The connector according to claim 121, wherein:
these ribs (66) are arranged periodically along the axis of the sleeve (65).

123. The connector according to claim 121, wherein:
the number of ribs (66) is between 5 and 15, the dimension of the recess as well as the hump of each rib (66) being between 0.5 mm and 2 mm.

124. The connector according to claim 120, wherein:
said sleeve (65) is permanently fixed to the guide tube (61) which it surrounds.

125. The connector according to claim 116, wherein:
the connector (60) comprises a lateral opening (67) enabling the insertion, into the connector (60), of another tube (87) coming from the arterial introducer (8).

126. A connection system comprising:
a connector (60) according to claim 116,
an arterial introducer (8),
a guide tube (61) for the elongate flexible medical instrument (1) of a robot for insertion of an elongate flexible medical instrument.

127. Connection system according to claim 126, wherein it comprises:
a catheter (1) and a guide (2) which are coaxial.

128. The connector according to claim 116, wherein:
the elongate flexible medical instrument is a catheter (1) and/or a guide (2).

129. A connection system comprising:
a connector (60) according to claim 117,
an arterial introducer (8),
a guide tube (61) for the elongate flexible medical instrument (1) of a robot for insertion of an elongate flexible medical instrument.

130. The connector according to claim 117, wherein:
the elongate flexible medical instrument is a catheter (1) and/or a guide (2).

131. The connector of claim 119, wherein the first fastener (63) comprises a plurality of longitudinal clips.

132. The connector of claim 131, wherein the second fastener (63) comprises a plurality of side clips.

133. The connector of claim 123, wherein the number of ribs is 10.

134. The connector of claim 133, wherein the dimension of the recess as well as the hump of each rib (66) is equal to 1 mm.

135. The connector of claim 123, wherein the dimension of the recess as well as the hump of each rib (66) is equal to 1 mm.
